SEQUENCE LISTING (42 sequences)

SEQ ID NO: 1 - protein, 254 aa, Homo sapiens
  SIGNAL (1)..(24)
  PROPEP (25)..(26): di-propeptide cleaved by cathepsin C
  mat_peptide (27)..(247): N-term sequence of active native proteinase 3 (PR3)
  PROPEP (248)..(254): C-terminal pro-peptide
RHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGP

SEQ ID NO: 2 - DNA, 1374 bp, Homo sapiens
  CDS (1)..(1374): 4C3 antibody, heavy chain
  V_region (1)..(381)
  V_segment (1)..(297)
  D-segment (310)..(330)
  J_segment (346)..(381)
  C_region (382)..(1374)
gaggtgcagctggtacagtcggggactgaggtgaagaagcctgggtcttcggtgaaggtctcctgcgagatttccggagacgacttcttcacctatggcgtcagctgggtgcggcaagcccctggacaaggacttgagtggatgggggggatcgtccccatgtttggtacaagaaactacgcacgtaaattccagggcagagtcacgattaccgcggacgactctacgagcacagtccacattgccctgaccagcctgacatttgacgacacggccatttattattgtgcgagagagggagtccccgactattgtactaatggtgtctgccactcctacgtcaaatactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - protein, 457 aa, Homo sapiens
  4C3 antibody, heavy chain
EVQLVQSGTEVKKPGSSVKVSCEISGDDFFTYGVSWVRQAPGQGLEWMGGIVPMFGTRNYARKFQGRVTITADDSTSTVHIALTSLTFDDTAIYYCAREGVPDYCTNGVCHSYVKYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 993 bp, Homo sapiens
  CDS (1)..(993): 4C3 antibody, heavy chain constant region
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 5 - protein, 330 aa, Homo sapiens
  4C3 antibody, heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 381 bp, Homo sapiens
  CDS (1)..(381): 4C3 antibody, heavy chain variable region
gaggtgcagctggtacagtcggggactgaggtgaagaagcctgggtcttcggtgaaggtctcctgcgagatttccggagacgacttcttcacctatggcgtcagctgggtgcggcaagcccctggacaaggacttgagtggatgggggggatcgtccccatgtttggtacaagaaactacgcacgtaaattccagggcagagtcacgattaccgcggacgactctacgagcacagtccacattgccctgaccagcctgacatttgacgacacggccatttattattgtgcgagagagggagtccccgactattgtactaatggtgtctgccactcctacgtcaaatactggggccagggaaccctggtcaccgtctcctca

SEQ ID NO: 7 - protein, 127 aa, Homo sapiens
  4C3 antibody, heavy chain variable region
EVQLVQSGTEVKKPGSSVKVSCEISGDDFFTYGVSWVRQAPGQGLEWMGGIVPMFGTRNYARKFQGRVTITADDSTSTVHIALTSLTFDDTAIYYCAREGVPDYCTNGVCHSYVKYWGQGTLVTVSS

SEQ ID NO: 8 - DNA, 297 bp, Homo sapiens
  CDS (1)..(297): 4C3 antibody, region V of the heavy chain variable region
gaggtgcagctggtacagtcggggactgaggtgaagaagcctgggtcttcggtgaaggtctcctgcgagatttccggagacgacttcttcacctatggcgtcagctgggtgcggcaagcccctggacaaggacttgagtggatgggggggatcgtccccatgtttggtacaagaaactacgcacgtaaattccagggcagagtcacgattaccgcggacgactctacgagcacagtccacattgccctgaccagcctgacatttgacgacacggccatttattattgtgcgagagag

SEQ ID NO: 9 - protein, 99 aa, Homo sapiens
  4C3 antibody, region V of the heavy chain variable region
EVQLVQSGTEVKKPGSSVKVSCEISGDDFFTYGVSWVRQAPGQGLEWMGGIVPMFGTRNYARKFQGRVTITADDSTSTVHIALTSLTFDDTAIYYCARE

SEQ ID NO: 10 - DNA, 21 bp, Homo sapiens
  CDS (1)..(21): 4C3 antibody, region D of the heavy chain variable region
tattgtactaatggtgtctgc

SEQ ID NO: 11 - protein, 7 aa, Homo sapiens
  4C3 antibody, region D of the heavy chain variable region
YCTNGVC

SEQ ID NO: 12 - DNA, 36 bp, Homo sapiens
  CDS (1)..(36): 4C3 antibody, region J of the heavy chain variable region
tactggggccagggaaccctggtcaccgtctcctca

SEQ ID NO: 13 - protein, 12 aa, Homo sapiens
  4C3 antibody, region J of the heavy chain variable region
YWGQGTLVTVSS

SEQ ID NO: 14 - DNA, 24 bp, Homo sapiens
  CDS (1)..(24): 4C3 antibody, CDR1 heavy chain
ggagacgacttcttcacctatggc

SEQ ID NO: 15 - protein, 8 aa, Homo sapiens
  4C3 antibody, CDR1 heavy chain
GDDFFTYG

SEQ ID NO: 16 - DNA, 24 bp, Homo sapiens
  CDS (1)..(24): 4C3 antibody, CDR2 heavy chain
atcgtccccatgtttggtacaaga

SEQ ID NO: 17 - protein, 8 aa, Homo sapiens
  4C3 antibody, CDR2 heavy chain
IVPMFGTR

SEQ ID NO: 18 - DNA, 60 bp, Homo sapiens
  CDS (1)..(60): 4C3 antibody, CDR3 heavy chain
gcgagagagggagtccccgactattgtactaatggtgtctgccactcctacgtcaaatac

SEQ ID NO: 19 - protein, 20 aa, Homo sapiens
  4C3 antibody, CDR3 heavy chain
AREGVPDYCTNGVCHSYVKY

SEQ ID NO: 20 - DNA, 645 bp, Homo sapiens
  CDS (1)..(645): 4C3 antibody, light chain
  V_region (1)..(324)
  V_segment (1)..(285)
  J_segment (287)..(321)
  C_region (325)..(645)
gacatccagatgacccagtctcctcccaccctgtctgcttctgtaggagacagagtcaccatcacttgccgggccagtcagagtattagtgactggttggcctggtatcagcagaaaccagggaaacctcctcagctcctcatctataaggcgtctagtttagaaagtggggtcccatcaaggttcagcggcactggatctggggcagaattcactctcaccatcagcagcctgcagcctgacgattctgcaacctattattgccaacagtataatagttactctaacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 21 - protein, 214 aa, Homo sapiens
  4C3 antibody, light chain
DIQMTQSPPTLSASVGDRVTITCRASQSISDWLAWYQQKPGKPPQLLIYKASSLESGVPSRFSGTGSGAEFTLTISSLQPDDSATYYCQQYNSYSNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 324 bp, Homo sapiens
  CDS (1)..(324): 4C3 antibody, constant light chain region
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 23 - protein, 107 aa, Homo sapiens [sequence omitted]

SEQ ID NO: 24 - DNA, 321 bp, Homo sapiens
  CDS (1)..(321): 4C3 antibody, light chain variable region
gacatccagatgacccagtctcctcccaccctgtctgcttctgtaggagacagagtcaccatcacttgccgggccagtcagagtattagtgactggttggcctggtatcagcagaaaccagggaaacctcctcagctcctcatctataaggcgtctagtttagaaagtggggtcccatcaaggttcagcggcactggatctggggcagaattcactctcaccatcagcagcctgcagcctgacgattctgcaacctattattgccaacagtataatagttactctaacacttttggccaggggaccaagctggagatcaaa

SEQ ID NO: 25 - protein, 107 aa, Homo sapiens
  4C3 antibody, light chain variable region
DIQMTQSPPTLSASVGDRVTITCRASQSISDWLAWYQQKPGKPPQLLIYKASSLESGVPSRFSGTGSGAEFTLTISSLQPDDSATYYCQQYNSYSNTFGQGTKLEIK

SEQ ID NO: 26 - DNA, 285 bp, Homo sapiens
  CDS (1)..(285): 4C3 antibody, region V of the light chain variable region
gacatccagatgacccagtctcctcccaccctgtctgcttctgtaggagacagagtcaccatcacttgccgggccagtcagagtattagtgactggttggcctggtatcagcagaaaccagggaaacctcctcagctcctcatctataaggcgtctagtttagaaagtggggtcccatcaaggttcagcggcactggatctggggcagaattcactctcaccatcagcagcctgcagcctgacgattctgcaacctattattgccaacagtataatagttactct

SEQ ID NO: 27 - protein, 95 aa, Homo sapiens
  4C3 antibody, region V of the light chain variable region
DIQMTQSPPTLSASVGDRVTITCRASQSISDWLAWYQQKPGKPPQLLIYKASSLESGVPSRFSGTGSGAEFTLTISSLQPDDSATYYCQQYNSYS

SEQ ID NO: 28 - DNA, 36 bp, Homo sapiens
  CDS (1)..(36): 4C3 antibody, region J of the light chain variable region
aacacttttggccaggggaccaagctggagatcaaa

SEQ ID NO: 29 - protein, 12 aa, Homo sapiens
  4C3 antibody, region J of the light chain variable region
NTFGQGTKLEIK

SEQ ID NO: 30 - DNA, 18 bp, Homo sapiens
  CDS (1)..(18): 4C3 antibody, CDR1 light chain
cagagtattagtgactgg

SEQ ID NO: 31 - protein, 6 aa, Homo sapiens
  4C3 antibody, CDR1 light chain
QSISDW

SEQ ID NO: 32 - DNA, 9 bp, Homo sapiens
  CDS (1)..(9): 4C3 antibody, CDR2 light chain
aaggcgtct

SEQ ID NO: 33 - protein, 3 aa, Homo sapiens
  misc_feature (1)..(3): 4C3 antibody, CDR2 light chain
KAS

SEQ ID NO: 34 - DNA, 27 bp, Homo sapiens
  CDS (1)..(27): 4C3 antibody, CDR2 light chain
caacagtataatagttactctaacact

SEQ ID NO: 35 - protein, 9 aa, Homo sapiens
  4C3 antibody, CDR3 light chain
QQYNSYSNT

SEQ ID NO: 36 - DNA, 1374 bp, Homo sapiens
  CDS (1)..(1374): Recombinant 4C3 antibody (r4C3), heavy chain
  V_region (1)..(381)
  V_segment (1)..(297)
  D-segment (310)..(330)
  J_segment (346)..(381)
  C_region (382)..(1374)
gaggtgcagctggtacagtcggggactgaggtgaagaagcctgggtcttcggtgaaggtctcctgcgagatttccggagacgacttcttcacctatggcgtcagctgggtgcggcaagcccctggacaaggacttgagtggatgggggggatcgtccccatgtttggtacaagaaactacgcacgtaaattccagggcagagtcacgattaccgcggacgactctacgagcacagtccacattgccctgaccagcctgacatttgacgacacggccatttattattgtgcgagagagggagtccccgactattgtactaatggtgtctgccactcctacgtcaaatactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - protein, 457 aa, Homo sapiens
  Recombinant 4C3 antibody (r4C3), heavy chain
EVQLVQSGTEVKKPGSSVKVSCEISGDDFFTYGVSWVRQAPGQGLEWMGGIVPMFGTRNYARKFQGRVTITADDSTSTVHIALTSLTFDDTAIYYCAREGVPDYCTNGVCHSYVKYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 993 bp, Homo sapiens
  CDS (1)..(993): Recombinant 4C3 antibody (r4C3), heavy chain constant region
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 39 - protein, 330 aa, Homo sapiens
  Recombinant 4C3 antibody (r4C3), heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 15 aa, Artificial Sequence
  4C3_3.2 peptide
TFFCRPHNICTFVPR

SEQ ID NO: 41 - protein, 15 aa, Artificial Sequence
  4C3_4.1 peptide
GTQCLAMGWGRVGAH

SEQ ID NO: 42 - protein, 221 aa, Homo sapiens
IVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLR

(19) United States
(12) Patent Application Publication
(10) Pub. No.: US 2023/0203197 A1
(43) Pub. Date: Jun. 29, 2023

(54) NOVEL HUMAN THERAPEUTIC MONOCLONAL ANTIBODIES AND USES THEREOF

(71) Applicants: UNIVERSITE DE TOURS, TOURS CEDEX 1 (FR); CENTRE HOSPITALIER REGIONAL UNIVERSITAIRE DE TOURS, TOURS CEDEX 09 (FR); INSTITUT NATIONAL DE LA SANTE ET DE LA RECHERCHE MEDICALE (INSERM), PARIS (FR)

(72) Inventors: Roxane LEMOINE, SAINT ETIENNE DE CHIGNY (FR); Cyrille HOARAU, FONDETTES (FR); Eric MORELLO, JOUE-LES-TOURS (FR); Brice KORKMAZ, VEIGNE (FR); Yann GALLAIS, SAINT-REMY LES CHEVREUSE (FR)

(73) Assignees: UNIVERSITE DE TOUR, TOURS CEDEX 1 (FR); CENTRE HOSPITALIER REGIONAL UNIVERSITAIRE DE TOURS, TOURS CEDEX 09 (FR); INSTITUT NATIONAL DE LA SANTE ET DE LA RECHERCHE MEDICALE (INSERM), PARIS (FR)

(21) Appl. No.: 17/926,817

(22) PCT Filed: Oct. 16, 2020
(86) PCT No.: PCT/EP2020/079208

§ 371 Date: Nov. 21, 2022

(30) Foreign Application Priority Data
Oct. 18, 2019 (FR) FR1911722

(51) Int. Cl. C07K 16/40; A61P 9/14

(52) U.S. Cl. CPC C07K 16/40; A61P 9/14; A61K 39/00

(57) ABSTRACT

New human therapeutic monoclonal antibodies, which are directed against neutrophil proteinase 3. The monoclonal antibodies are specifically directed against a conformational epitope of the neutrophil proteinase 3 and are capable of inhibiting by at least 30% the production for reactive oxygen derivatives by neutrophils. Also, a pharmaceutical composition including as an active substance at least the monoclonal antibody, and a method for early treatment and/or prevention of relapse of granulomatosis with polyangiitis, which includes the administration of the pharmaceutical composition.

### FIELD OF INVENTION

The invention concerns new human therapeutic monoclonal antibodies and their uses.

### CONTEXT OF THE INVENTION

In granulomatosis with polyangiitis (GPA or “Wegener's” disease), neutrophil proteinase 3 (PR3) is the target of autoantibodies (ANCA) which leads to a deleterious activation of neutrophil polymorphisms (PMN), responsible for severe vasculitis, affecting mainly the kidneys, lungs and brain. Current treatments for GPA are based on immunosuppressive drugs, corticosteroids, and more recently anti-CD20 antibodies. There is no precise targeting of the PMNs but a global action on the inflammation and on the immune cells. Anti-CD20 targets more specifically circulating B lymphocytes expressing the CD20 differentiation marker. This results in a deletion of B lymphocytes producing autoantibodies directed against PR3 of PMN (ANCA). However, this deletion is neither immediate nor without any side effects on immunity. Indeed, anti-CD20 deletes the circulating B-lymphocytes but has no direct effect on the plasma cells that produce the antibodies. It is therefore necessary to wait several weeks to see a decrease in ANCA. Furthermore, anti-CD20s do not specifically target ANCA-producing B cells, but all B cells. The deletion of B lymphocytes is thus global and can induce a decrease in the overall immunoglobulins, which can lead to a humoral immune deficiency with risks of serious infections. This risk increases with the use of other immunosuppressive treatments, which are usually prescribed for several months.

Current therapeutic treatments are therefore not effective or even harmful to the patient and many medical needs remain unresolved to this day. These include the need to obtain a more rapidly effective treatment, and to reduce the duration and intensity of immunosuppressive treatments responsible for serious side effects.

### BRIEF OVERVIEW OF THE INVENTION

In this context of a search for suitable and effective therapeutic tools, and thus to make up for the current shortfalls, a first aim of the invention is to propose an antibody targeting neutrophil proteinase 3 (PR3). A second aim of the invention is to propose fragments of this antibody. A third purpose of the invention is to provide the tools (nucleic acid, vector, etc.) to produce said antibody and/or said fragments. Finally, another purpose of the invention is to propose pharmaceutical compositions and their uses.

### DETAILED DESCRIPTION

The present invention relates to the object as defined and described hereafter. In addition, and unless otherwise specified or the context otherwise requires, all terms have their ordinary meaning in the relevant field(s).

According to a first aspect of the invention, the object of the invention is a monoclonal antibody directed against neutrophil proteinase 3 represented by the sequence SEQ ID NO: 1, the said monoclonal antibody:
being specifically directed against a conformational epitope of said neutrophil proteinase 3; and
being capable of inhibiting by at least 30% the production of reactive oxygen derivatives by neutrophils,
said production of reactive oxygen derivatives being induced by the presence of autoantibodies directed against said neutrophil proteinase 3.

In the invention, the term “antibody” refers to an immunoglobulin, a multimeric protein consisting of 4 chains participating in the acquired immune response.

Immunoglobulins are well known to the man in the trade and are made up of an assembly of two dimers, each consisting of a heavy chain and a light chain. The multimeric complex is assembled by linking a light chain and a heavy chain by a disulphide bridge between two cysteines, the two heavy chains being themselves also linked by two disulphide bridges.

Each of the heavy and light chains consists of a constant region and a variable region. The assembly of the chains that make up an antibody defines a characteristic three-dimensional Y-shaped structure, where
the base of the Y corresponds to the constant region Fc which is recognised by the complement and Fc receptors, and
The ends of the Y-arms correspond to the respective assembly of the variable regions of the light and variable regions of the heavy chain.

More precisely, each light chain is made up of a variable region (V_{L}) and a constant region (C_{L}). Each heavy chain consists of a variable region (V_{H}) and a constant region consisting of three constant domains C_{H}1, C_{H}2 and C_{H}3. The C_{H}2 and C_{H}3 domains make up the Fc domain.

The variable region of the light chain consists of three antigen recognition determining regions (CDRs) surrounded by four framework domains. The heavy chain variable region also consists of three CDRs surrounded by four framework domains. The three-dimensional folding of these variable regions is such that the 6 CDRs are exposed on the same side of the protein and allow the formation of a specific structure recognising a given antigen.

The antibodies described in the invention are isolated and purified, may be recombinant, may belong to any isotype/class (e.g. IgG, IgE, IgM, IgD, IgA and IgY) or subclass (e.g. IgG1, IgG2, IgG3, IgG4, IgA1 and IgA2) and are different from natural antibodies. These antibodies are mature, i.e. they have an ad hoc three-dimensional structure enabling them to recognise the antigen, and possess all the post-translational modifications essential for antigen recognition, including glycosylation and the formation of intra- and intermolecular disulphide bridges.

More specifically, these are “monoclonal antibodies”, i.e. they recognise only one antigenic determinant of neutrophil proteinase 3 (PR3), unlike polyclonal antibodies which correspond to a mixture of antibodies, and can therefore recognise several antigenic determinants of the same protein. In particular, it should be noted that the monoclonal antibodies of the invention specifically target a “conformational epitope” of PR3, which is formed/constituted by amino acids which are not contiguous in the sequence SEQ ID NO: 1, however, some of said amino acids may be immediately adjacent to each other and thus a conformational epitope according to the present invention may contain only one amino acid which is not contiguous to the others in the sequence SEQ ID NO: 1.

In the invention, the expression “production of reactive oxygen species” (ROS) refers in particular to the cellular mechanisms enabling neutrophils to produce oxygenated chemical species such as free radicals, oxygen ions and peroxides, made chemically highly reactive by the presence of unpaired valence electrons. These can be, for example, the superoxide anion O₂⁻, singlet oxygen O₂·, hydrogen peroxide H₂O₂, or ozone O₃, which can, for example, be detected by flow cytometry using the DHR123-FITC (hydrogen peroxide H₂O₂ detection) label (see Examples, point 14). In particular, it should be noted that the monoclonal antibodies of the invention are capable of “inhibiting by at least 30%” this production of reactive oxygen derivatives by neutrophils, which activate in the presence of auto-antibodies (ANCA) directed against the said proteinase 3 of the neutrophil. In addition and in the sense of the invention, the expression “at least 30%” must be understood as being at least 35%, at least 40%, at least 45%, at least 50%, at least 55%, at least 60%, at least 65%, at least 70% or at least 75%. This may also mean that the ability of the antibodies of the invention to inhibit the said production of reactive oxygen derivatives may range from 30% to 80% or from 36% to 71%.

Since the monoclonal antibody (recombinant and/or isolated and purified) of the invention (as defined above) advantageously possesses this unexpected feature of particular inhibition, the invention responds to the issues raised. Indeed, the action of an antibody being immediate on circulating or endothelial cells, in GPA, where the PMN target is the endothelial cell, the monoclonal antibody of the invention:
causes an immediate and specific competitive inhibition of the PR3/ANCA interaction, and
allows to quickly and effectively block/inhibit/neutralise the deleterious activation of PMN (e.g. production of reactive oxygen derivatives) and thus the resulting deleterious inflammation.

By limiting endothelial lesions from the very first days of treatment, this effect also has the advantage of reducing the risk of after-effects, particularly renal, pulmonary and cerebral ones. Furthermore, it should be noted that the expression “monoclonal antibody (recombinant and/or isolated and purified)” means that the monoclonal antibody of the invention can be:
recombinant;
isolated and purified; or
recombinant, isolated and purified.

According to another mode of realization, the invention has as its object the monoclonal antibody (recombinant and/or isolated and purified) as previously defined comprising:
a heavy chain comprising from its N-terminal to its end C-terminal:
   a CDR1 having at least 80% identity with the sequence SEQ ID NO: 15;
   a CDR2 having at least 80% identity with the sequence SEQ ID NO: 17; and
   a CDR3 having at least 80% identity with the sequence SEQ ID NO: 19; and
a light chain comprising from its N-terminal to its end C-terminal:
   a CDR1 having at least 80% identity with the sequence SEQ ID NO: 31;
   a CDR2 having at least 80% identity with the sequence SEQ ID NO: 33; and
   a CDR3 having at least 80% identity with the sequence SEQ ID NO: 35.

In the sense of the invention, this sequence identity between an amino acid sequence of interest and a reference amino acid sequence (e.g. CDR1 of the heavy chain; SEQ ID NO: 15) being at least 80%, this means that it may be at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%. In particular, it is at least 93%. The measurement of this sequence identity, as well as of all those subsequently described in the invention, are measured by the classic tools for comparing sequences known to the skilled person, such as the algorithms of the BLAST platform or, preferably, the MatGat2.01 programme under the BLOSUM 50 algorithm (Campanella, J. J., Bitincka, L., & Smalley, J. (2003). MatGAT: an application that generates similarity/identity matrices using protein or DNA sequences. BMC Bioinformatics, 4, 29.).

According to another mode of realization, the invention has as its object the monoclonal antibody (recombinant and/or isolated and purified) as previously defined comprising:
a heavy chain comprising from its N-terminal to its end C-terminal:
   the CDR1 of sequence SEQ ID NO: 15;
   the CDR2 of sequence SEQ ID NO: 17; and
   the CDR3 of sequence SEQ ID NO: 19; and
a light chain comprising from its N-terminal to its end C-terminal:
   the CDR1 of sequence SEQ ID NO: 31;
   the CDR2 of sequence SEQ ID NO: 33; and
   the CDR3 of sequence SEQ ID NO: 35.

According to another mode of realization, the invention has as its object the monoclonal antibody (recombinant and/or isolated and purified) as previously defined comprising:
a heavy chain comprising a variable region having at least 80% identity with the sequence SEQ ID NO: 7, with the proviso that said variable region of the heavy chain comprises from its N-terminal end to its C-terminal end:
   the CDR1 of sequence SEQ ID NO: 15;
   the CDR2 of sequence SEQ ID NO: 17; and
   the CDR3 of sequence SEQ ID NO: 19; and
a light chain comprising a variable region having at least 80% identity with the sequence SEQ ID NO: 25, with the proviso that said variable region of the light chain comprises from its N-terminal end to its C-terminal end:
   the CDR1 of sequence SEQ ID NO: 31;
   the CDR2 of sequence SEQ ID NO: 33; and
   the CDR3 of sequence SEQ ID NO: 35.

According to another mode of realization, the invention has as its object the monoclonal antibody (recombinant and/or isolated and purified) as previously defined comprising:
a heavy chain comprising the sequence variable region SEQ ID NO: 7; and
a light chain comprising the sequence variable region SEQ ID NO: 25.

According to another mode of realization, the invention has as its object the monoclonal antibody (recombinant and/or isolated and purified) as previously defined comprising:
a heavy chain comprising or consisting of a sequence having at least 80% identity with the sequence SEQ ID NO: 3, with the proviso that said heavy chain comprises the variable region of sequence SEQ ID NO: 7; and
a light chain comprising or consisting of a sequence having at least 80% identity with the sequence SEQ ID NO: 21, with the proviso that said light chain comprises the variable region of sequence SEQ ID NO: 25.

In particular, the invention relates to the monoclonal antibody as previously defined comprising:
a heavy chain comprising or consisting of the sequence SEQ ID NO: 3 or the sequence SEQ ID NO: 37; and
a light chain comprising or consisting of the sequence SEQ ID NO: 21.

More precisely, this method of production corresponds to the so-called “4C3” antibody of the G1m3-1 allotype (SEQ ID NOs: 3 and 21) or to its recombinant “r4C3” allotype. G1m17-1 (SEQ ID NOs: 37 and 21) and whose set of sequences is in Table 1 below, which differ from each other by only one mutation, namely, R>K in position 224 (or AGA>AAA in position 670-672) within the constant region of the heavy chain.

TABLE 1
Sequences of 4C3 and r4C3antibodies

| | | 4C3 | | r4C3 | |
|---|---|---|---|---|---|
| | | Nucleic | Amino | Nucleic | Amino |
| | | acid SEQ | acid SEQ | acid SEQ | acid SEQ |
| | | ID NO: | ID NO: | ID NO: | ID NO: |
| Heavy | Complete | 2 | 3 | 36 | 37 |
| chain | Constant region | 4 | 5 | 38 | 39 |
| | Variable region | 6 | 7 | 6 | 7 |
| | Region V | 8 | 9 | 8 | 9 |
| | Region D | 10 | 11 | 10 | 11 |
| | Region J | 12 | 13 | 12 | 13 |
| | CDR1 | 14 | 15 | 14 | 15 |
| | CDR2 | 16 | 17 | 16 | 17 |
| | CDR3 | 18 | 19 | 18 | 19 |
| Light | Complete | 20 | 21 | 20 | 21 |
| chain | Constant region | 22 | 23 | 22 | 23 |
| | Variable region | 24 | 25 | 24 | 25 |
| | Region V | 26 | 27 | 26 | 27 |
| | Region J | 28 | 29 | 28 | 29 |
| | CDR1 | 30 | 31 | 30 | 31 |
| | CDR2 | 32 | 33 | 32 | 33 |
| | CDR3 | 34 | 35 | 34 | 35 |

According to another particular mode of realization, the invention has as its object the monoclonal antibody (recombinant and/or isolated and purified) as previously defined comprising:
a heavy chain comprising from its N-terminal to its end C-terminal:
   a CDR1 having at least 80% identity with the sequence SEQ ID NO: 15;
   a CDR2 having at least 80% identity with the sequence SEQ ID NO: 17; and
   a CDR3 having at least 80% identity with the sequence SEQ ID NO: 19; and
a light chain comprising from its N-terminal to its end C-terminal:
   a CDR1 having at least 80% identity with the sequence SEQ ID NO: 31;
   a CDR2 having at least 80% identity with the sequence SEQ ID NO: 33; and
   a CDR3 having at least 80% identity with the sequence SEQ ID NO: 35, in particular including:
a heavy chain comprising a variable region having at least 80% identity with the sequence SEQ ID NO: 7, with the proviso that said variable region of the heavy chain comprises from its N-terminal end to its C-terminal end:
   the CDR1 of sequence SEQ ID NO: 15;
   the CDR2 of sequence SEQ ID NO: 17; and
   the CDR3 of sequence SEQ ID NO: 19; and
a light chain comprising a variable region having at least 80% identity with the sequence SEQ ID NO: 25, with the proviso that said variable region of the light chain comprises from its N-terminal end to its C-terminal end:
   the CDR1 of sequence SEQ ID NO: 31;
   the CDR2 of sequence SEQ ID NO: 33; and
   the CDR3 of sequence SEQ ID NO: 35, and preferably including:
a heavy chain comprising or consisting of a sequence having at least 80% identity with the sequence SEQ ID NO: 3, with the proviso that said heavy chain comprises the variable region of sequence SEQ ID NO: 7; and
a light chain comprising or consisting of a sequence having at least 80% identity with the sequence SEQ ID NO: 21, with the proviso that said light chain comprises the variable region of sequence SEQ ID NO: 25.

According to a second aspect of the invention, the subject matter of the invention is a fragment of a monoclonal antibody (recombinant and/or isolated and purified) as described above. In the invention, the term “fragment” refers to any part of an antibody which retains the ability to bind to the epitope recognised by the complete antibody. Examples of such fragments include, but are not limited to, Fab, Fab′ and F(ab′)₂, Fd, single chain Fvs (scFv), single chain antibodies, disulphide bonded Fvs (dsFv) and fragments comprising the V_{L} or V_{H} region. Fragments binding to the epitope, including single chain antibodies, may comprise the variable region(s) alone or in combination with all or some of the following: hinge region, C_{H}1, C_{H}2 and C_{H}3 domains.

Such fragments may contain one or both Fab fragments or the F(ab′)₂ fragment. In addition, the fragments may be or may combine members of any of the following immunoglobulin classes: IgG, IgM, IgA, IgD or IgE and their subclasses.

The Fab and F(ab′)₂ fragments can be produced by proteolytic cleavage, using enzymes such as papain (Fab fragment) or pepsin (F(ab′)₂ fragment).

Single chain Fv fragments (“scFv”) are epitope-binding fragments that contain at least one fragment of an antibody variable region (V_{H}) linked to at least one fragment of a light chain antibody variable region (V_{L}). The linker may be a short, flexible peptide selected to ensure that correct three-dimensional folding of the V_{L} and V_{H} regions occurs once they are bound, so as to maintain the binding specificity to the target molecule of the whole antibody from which the single-chain antibody fragment is derived. The carboxyl end of the V_{L} or V_{H} sequence may be covalently linked by a binding agent to the amino acid end of a complementary V_{L} or V_{H} sequence.

According to a particular mode of realisation of this second aspect, the invention relates to the above fragment of a monoclonal antibody (recombinant and/or isolated and purified) as defined above, said fragment being selected from the group of fragments consisting of: Fv, Fab, F(ab′)₂, Fab′, dsFv, scFv, sc(Fv)₂, “diabodies”.

This being the case, it is understood in the sense of the invention that the subject of the invention is the above fragment of a monoclonal antibody (recombinant and/or isolated and purified) as defined above, the said fragment comprising the combination of the six (6) CDRs having at least 80% identity with the sequences SEQ ID NOs; 15, 17, 19, 31, 33 and 35.

In the sense of the invention, this sequence identity between an amino acid sequence of interest and a reference amino acid sequence (e.g. CDR1 of the heavy chain; SEQ ID NO: 15) being at least 80%, this means that it may be at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%. In particular, it is at least 93%. The measurement of this sequence identity, as well as of all those subsequently described in the invention, are measured by the classic tools for comparing sequences known to the skilled person, such as the algorithms of the BLAST platform or, preferably, the MatGat2.01 programme under the BLOSUM 50 algorithm (Campanella, J. J., Bitincka, L., & Smalley, J. (2003). MatGAT: an application that generates similarity/identity matrices using protein or DNA sequences. BMC Bioinformatics, 4, 29.).

According to another mode of realization, the invention relates to the above fragment of a monoclonal antibody (recombinant and/or isolated and purified) as previously defined, said fragment comprising the combination of the six (6) CDRs of sequences SEQ ID NOs; 15, 17, 19, 31, 33 and 35.

According to another mode of realization, the invention relates to the above fragment of a monoclonal antibody (recombinant and/or isolated and purified) as defined above, said fragment comprising:
a variable region of a heavy chain having at least 80% identity with the sequence SEQ ID NO: 7, with the proviso that said variable region of the heavy chain comprises from its N-terminal end to its C-terminal end:
   the CDR1 of sequence SEQ ID NO: 15;
   the CDR2 of sequence SEQ ID NO: 17; and
   the CDR3 of sequence SEQ ID NO: 19; and
a variable region of a light chain having at least 80% identity with the sequence SEQ ID NO: 25, with the proviso that said variable region of the light chain comprises from its N-terminal end to its C-terminal end:
   the CDR1 of sequence SEQ ID NO: 31;
   the CDR2 of sequence SEQ ID NO: 33; and
   the CDR3 of sequence SEQ ID NO: 35.

According to another mode of realization, the invention relates to the above fragment of a monoclonal antibody (recombinant and/or isolated and purified) as defined above, said fragment comprising:
a variable region of a heavy chain sequence SEQ ID NO: 7; and
a variable region of a light chain sequence SEQ ID NO: 25.

According to a third aspect of the invention, the subject matter of the invention is a nucleic acid comprising or consisting of a coding sequence:
the monoclonal antibody (recombinant and/or isolated and purified) as defined above; or
the heavy chain of a monoclonal antibody (recombinant and/or isolated and purified) as defined above and/or the light chain of a monoclonal antibody (recombinant and/or isolated and purified) as defined above; or
the fragment as defined above.

According to another mode of realisation of this third aspect, the invention relates to nucleic acid as defined above comprising or consisting of a coding sequence:
a heavy chain comprising from its N-terminal to its end C-terminal:
   a CDR1 having at least 80% identity with the sequence SEQ ID NO: 14;
   a CDR2 having at least 80% identity with the sequence SEQ ID NO: 16; and
   a CDR3 having at least 80% identity with the sequence SEQ ID NO: 18; and/or
a light chain comprising from its N-terminal to its end C-terminal:
   a CDR1 having at least 80% identity with the sequence SEQ ID NO: 30;
   a CDR2 having at least 80% identity with the sequence SEQ ID NO: 32; and
   a CDR3 having at least 80% identity with the sequence SEQ ID NO: 34.

In the sense of the invention, this sequence identity between a nucleic acid sequence of interest and a reference nucleic acid sequence (e.g., a nucleic acid sequence of interest and a reference nucleic acid sequence) is a prerequisite for the invention. CDR1 of the heavy chain; SEQ ID NO: 14) being at least 80%, this means that it may be at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%. In particular, it is at least 93%. The measurement of this sequence identity, as well as of all those subsequently described in the invention, are measured by the classic tools for comparing sequences known to the skilled person, such as the algorithms of the BLAST platform or preferably the MatGat2.01 programme (Campanella, J. J., Bitincka, L., & Smalley, J. (2003). MatGAT: an application that generates similarity/identity matrices using protein or DNA sequences. BMC Bioinformatics, 4, 29.).

According to another mode of realisation of this third aspect, the invention relates to nucleic acid as defined above comprising or consisting of a coding sequence:
a heavy chain comprising from its N-terminal to its end C-terminal:
   the CDR1 of sequence SEQ ID NO: 14;
   the CDR2 of sequence SEQ ID NO: 16; and
   the CDR3 of sequence SEQ ID NO: 18; and/or
a light chain comprising from its N-terminal to its end C-terminal:
   the CDR1 of sequence SEQ ID NO: 30;
   the CDR2 of sequence SEQ ID NO: 32; and
   the CDR3 of sequence SEQ ID NO: 34.

According to another mode of realisation of this third aspect, the invention relates to nucleic acid as defined above comprising or consisting of a coding sequence:
a heavy chain comprising a variable region having at least 80% identity with the sequence SEQ ID NO: 6, with the proviso that said variable region of the heavy chain comprises from its N-terminal end to its C-terminal end:
   the CDR1 of sequence SEQ ID NO: 14;
   the CDR2 of sequence SEQ ID NO: 16; and
   the CDR3 of sequence SEQ ID NO: 18; and/or
a light chain comprising a variable region having at least 80% identity with the sequence SEQ ID NO: 24, with the proviso that said variable region of the light chain comprises from its N-terminal end to its C-terminal end:
   the CDR1 of sequence SEQ ID NO: 30;
   the CDR2 of sequence SEQ ID NO: 32; and
   the CDR3 of sequence SEQ ID NO: 34.

According to another mode of realisation of this third aspect, the invention relates to nucleic acid as defined above comprising or consisting of a coding sequence:
a heavy chain comprising the sequence variable region SEQ ID NO: 6; and/or
a light chain comprising the sequence variable region SEQ ID NO: 24.

According to another mode of realisation of this third aspect, the invention relates to nucleic acid as defined above comprising or consisting of a coding sequence:
a heavy chain comprising or consisting of a sequence having at least 80% identity with the sequence SEQ ID NO: 2, with the proviso that said heavy chain comprises the variable region of sequence SEQ ID NO: 6; and/or
a light chain comprising or consisting of a sequence having at least 80% identity with the sequence SEQ ID NO: 20, with the proviso that said light chain comprises the variable region of sequence SEQ ID NO: 24.

In particular, the invention relates to nucleic acid as previously defined comprising or consisting of a coding sequence:
a heavy chain comprising or consisting of the sequence SEQ ID NO: 2 or the sequence SEQ ID NO: 36; and/or
a light chain comprising or consisting of the sequence SEQ ID NO: 20.

More precisely, this method of production corresponds to the so-called “4C3” antibody of the G1m3-1 allotype (SEQ ID NOs: 2 and 20) or to its recombinant “r4C3” allotype. G1m17-1 (SEQ ID NOs: 36 and 20) and whose set of sequences is in Table 1 above, which differ from each other by only one mutation, namely, R>K in position 224 (or AGA>AAA in position 670-672) within the constant region of the heavy chain.

According to another particular mode of realisation of this third aspect, the object of the invention is an expression vector comprising at least one nucleic acid as defined above, said nucleic acid being under the control of elements allowing its expression.

The term “expression vector” is defined in the invention as a DNA molecule (deoxyribonucleic acid) that possesses elements that enable it to be replicated (duplicated) in at least one living organism. These elements enabling replication are in particular yeast or bacterial replication origins, or elements controlling the replication of a virus.

The vectors according to the invention are in particular plasmids, phages, artificial chromosomes of yeast (YAC), artificial chromosomes of bacteria (BAC), modified genomes of replicative or integrative viruses, etc.

These vectors are called “expression” vectors because they have nucleotide sequences that allow the expression, i.e. transcription into RNA (ribonucleic acid), of the nucleotide sequences they control.

In the invention, the said nucleic acid sequence contained in the said vector is placed “under the control of the elements allowing its expression”. This means that said expression vector has at least one transcription initiation sequence such as a virus promoter such as the early promoter of the simian virus SV40, or of the Cytomegalovirus (CMV) or the promoter sequences of the Rous sarcoma virus (RSV), and in particular a sequence or promoter comprising a TATAA box. In addition, said vector also has at least one transcription termination sequence and in particular a polyadenylation sequence from a mammalian gene, in particular a human gene.

To these sequences, which are indispensable for the expression of the nucleotide sequence contained in the said vector, other sequences may be added which make it possible to regulate or modulate the expression of the said sequence. A non-exhaustive list includes: introns of mammalian genes, in particular human genes, enhancer-type transcription regulation sequences (“enhancers”) or transcribed but untranslated sequences of mammalian genes, in particular human genes.

A particular mode of realization of the invention therefore has as its object a vector of expression as previously defined, comprising:
a first nucleic acid selected from those encoding all or part of the heavy chain of the monoclonal antibody as described above, said first nucleic acid being under the control of the elements permitting its expression; and
a second nucleic acid selected from those encoding all or part of the light chain of the monoclonal antibody as described above, said second nucleic acid being under the control of the elements permitting its expression.

This expression vector therefore comprises two nucleic acid sequences as mentioned above, and more specifically comprises a nucleic acid sequence encoding the heavy chain of the monoclonal antibody as described above, and a nucleic acid sequence encoding the light chain of the monoclonal antibody as described above.

Preferentially, said expression vector contains a first element allowing the expression of the nucleic acid sequence encoding the heavy chain of the monoclonal antibody (recombinant and/or isolated and purified) as described above and a second element allowing the expression of the nucleic acid sequence encoding the light chain of the monoclonal antibody (recombinant and/or isolated and purified) as described above, said first and said second element allowing the expression of said nucleic acid sequences being identical or different, and preferably identical. These control elements are in particular the long terminal repeated sequences (LTR) of the RSV virus.

According to another particular mode of realisation of this third aspect, the invention relates to a host cell or cell line transformed by a nucleic acid as described above and/or an expression vector as described above.

According to a fourth aspect of the invention, the purpose of the invention is the use of at least:
a monoclonal antibody (recombinant and/or isolated and purified) as described above; and/or
a fragment as described above; and/or
a nucleic acid as described above; and/or
an expression vector as described above,
   as a medicine. In other words, the invention relates to the monoclonal antibody (recombinant and/or isolated and purified) as previously described and/or fragment as previously described and/or nucleic acid as previously described and/or expression vector as previously described for its use as a medicament.

According to another way of realising this fourth aspect, the invention has as its object a pharmaceutical composition comprising at least one active substance:
a monoclonal antibody (recombinant and/or isolated and purified) as described above; and/or
a fragment as described above; and/or
a nucleic acid as described above; and/or
an expression vector as described above,
   in combination with a pharmaceutically acceptable vehicle.

For the purposes of the invention, “pharmaceutical composition” means a particular packaging of the invention, which allows the pharmaceutical composition of the invention to be possibly administered to animals and human beings by oral, sublingual, subcutaneous, intramuscular, intravenous, transdermal, local, inhaled or rectal route. Moreover, this packaging also allows the administration of the active principle (or active substance), alone or in combination with another active principle, in unitary form or in mixture with conventional pharmaceutical carriers. Appropriate unitary forms of administration include:
oral forms of administration such as tablets, capsules, powders, granules and oral suspensions or solutions;
sublingual and oral forms of administration, aerosols, implants . . . ;
subcutaneous, transdermal, intradermal, intraperitoneal, intramuscular, intravenous, subcutaneous, transdermal, intratracheal and nasal administration forms; and
rectal administration forms.

For the purposes of the invention, “pharmaceutically acceptable vehicle” means a non-toxic material that is compatible with a biological system such as a cell, cell culture, tissue or an animal or human organism. This may include, in particular:
crystalloid solutes, e.g. sodium chloride, bicarbonate, glucose;
of cationic lipids;
peptide compounds; or
of surfactants, e.g. polysorbates.

In all cases, whatever formulation is chosen, it must be sterile, stable under manufacturing and storage conditions, and must imperatively be preserved from any contamination by micro-organisms, such as bacteria and fungi.

According to another way of realising this fourth aspect, the invention has as its object a pharmaceutical composition comprising at least one active substance:
a monoclonal antibody (recombinant and/or isolated and purified) as described above, said monoclonal antibody being at a dose of between 5 mg to 1,000 mg, preferably from 100 mg to 800 mg; and/or
a fragment as described above, said fragment being at a dose of between 5 mg and 1,000 mg, preferably between 100 mg and 800 mg; and/or
a nucleic acid as described above, said nucleic acid being at a dose of between 5 mg and 1,000 mg, preferably between 100 mg and 800 mg; and/or
an expression vector as described above, said expression vector being at a dose of 5 mg to 1,000 mg, preferably 100 mg to 800 mg,
   in combination with a pharmaceutically acceptable vehicle.

According to another particular mode of realisation of this fourth aspect, the invention has as its object a pharmaceutical composition comprising at least one active substance:
a monoclonal antibody (recombinant and/or isolated and purified) as described above; and/or
a fragment as described above; and/or
a nucleic acid as described above; and/or
an expression vector as described above,
   in combination with a pharmaceutically acceptable vehicle,
   in particular,
said monoclonal antibody (recombinant and/or isolated and purified) as described above being at a dose of 5 mg to 1,000 mg; and/or
said fragment as described above being at a dose of between 5 mg and 1,000 mg; and/or
said nucleic acid as described above being at a dose of between 5 mg and 1,000 mg; and/or
said expression vector as described above being at a dose of 5 mg to 1,000 mg.

For the purposes of the invention, the expression “dose from 5 mg to 1,000 mg” means that the dose may be from 50 mg to 900 mg, from 100 mg to 800 mg, from 200 mg to 700 mg, from 300 mg to 600 mg, from 400 mg to 500 mg or from 5 mg to 500 mg. It also means that the dose can be 5 mg, 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg or 1,000 mg.

According to another mode of realisation of this fourth aspect, the invention has as its object a pharmaceutical composition as described above, for its use in the early treatment and/or prevention of relapse of granulomatosis flare-ups with polyangiitis.

The invention aims at providing a tool for the early treatment and/or prevention of relapse in the context of granulomatosis with polyangiitis, which is a severe systemic necrotizing vasculitis, i.e. inflammation with necrosis of the vascular walls, the expression “early treatment and/or prevention of relapse” is understood in the sense of the invention as being the use of the antibody, antibody fragment, nucleic acid or expression vector of the invention as described above in a patient as soon as the first clinical signs of the pathology and/or relapse appear, which are known to the person skilled in the art and may be, for example:
Muscle and/or joint pain;
Mild fever;
nosebleed;
abnormally coloured urine;
High blood pressure;
Caughing up blood;
swellings in the limbs;
etc.

Furthermore, “early” in the sense of the invention means the use of the antibody, antibody fragment, nucleic acid or expression vector of the invention as described above as soon as the diagnosis of the pathology is made, said diagnosis being able to be made in particular by searching for biological markers such as ANCA auto-antibodies in a blood test for example. Similarly, “relapse” in the sense of the invention is understood as the reappearance of the clinical signs of the pathology (see above) after the use of an adapted treatment (e.g. the antibody of the invention) which has worked following the first appearance of the pathology. The diagnosis of these relapses is therefore generally quicker to make, as the disease is already known. Also and in view of the foregoing, the expression “early treatment and/or prevention of relapse” may be understood as being the use (i.e. the implementation of a specific or prolonged therapy) of the antibody, antibody fragment, nucleic acid or expression vector of the invention as described above in a patient within the first two (2) to four (4) weeks following the appearance of the first clinical signs of the pathology and/or relapse.

According to another mode of realisation of this fourth aspect, the invention has as its object a pharmaceutical composition for its use as described above, said pharmaceutical composition further comprising anti-inflammatory agents, such as corticoids, and/or immunosuppressants.

According to another mode of realisation of this fourth aspect, the invention has as its object a pharmaceutical composition for its use as described above, in which:
said active substance is a monoclonal antibody (recombinant and/or isolated and purified) as described above, said monoclonal antibody being at a dose ranging from 1 mg/kg to 50 mg/kg, preferably from 5 mg/kg to 25 mg/kg; and/or
said active substance is a fragment as described above, said fragment being at a dose of 1 mg/kg to 50 mg/kg, preferably 5 mg/kg to 25 mg/kg; and/or
said active substance is a nucleic acid as described above, said nucleic acid being at a dose ranging from 1 mg/kg to 50 mg/kg, preferably from 5 mg/kg to 25 mg/kg; and/or
said active substance is an expression vector as described above, said expression vector being at a dose ranging from 1 mg/kg to 50 mg/kg, preferably from 5 mg/kg to 25 mg/kg.

For the purposes of the invention, the expression “dose from 1 mg/kg to 50 mg/kg” means that the dose may be from 5 mg/kg to 45 mg/kg, from 10 mg/kg to 40 mg/kg, from 15 mg/kg to 35 mg/kg, from 20 mg/kg to 30 mg/kg, from 25 mg/kg to 50 mg/kg or from 1 mg/kg to 10 mg/kg. This also means that the dose can be 1 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg or 50 mg/kg.

According to another mode of realisation of this fourth aspect, the invention has as its object a unitary pharmaceutical composition for its use as described above, in which:
said active substance is a monoclonal antibody (recombinant and/or isolated and purified) as described above, said monoclonal antibody being at a dose of between 70 mg and 3,500 mg per unit dose (based on a 70 kg Male), preferably between 1,000 mg and 2,500 mg per unit dose (based on a 70 kg Male); and/or
said active substance is a fragment as described above, said fragment being at a dose of between 70 mg and 3,500 mg per unit dose (based on a 70 kg Male), preferably between 1,000 mg and 2,500 mg per unit dose (based on a 70 kg Male); and/or
said active substance is a nucleic acid as described above, said nucleic acid being at a dose of between 70 mg and 3,500 mg per unit dose (based on a 70 kg human), preferably between 1,000 mg and 2,500 mg per unit dose (based on a 70 kg human); and/or
the said active substance is an expression vector as described above, the said expression vector being at a dose of between 70 mg and 3,500 mg per unit dose (based on a 70 kg man), preferably between 1,000 mg and 2,500 mg per unit dose (based on a 70 kg man).

For the purposes of the invention, the expression “dose from 70 mg to 3500 mg per unit dose” means that the dose may be from 100 mg to 3000 mg, from 500 mg to 2500 mg, from 1000 mg to 2000 mg, from 1500 mg to 3500 mg or from 70 mg to 1500 mg per unit dose. It also means that the dose can be 70 mg, 100 mg, 500 mg, 1,000 mg, 1,500 mg, 2,000 mg, 2,500 mg, 3,000 mg or 3,500 mg per unit dose.

According to another mode of realisation of this fourth aspect, the invention relates to a pharmaceutical composition for its use as described above, said composition being formulated to be administered by one of the following routes: oral, parenteral, injectable, topical, inhalation, subcutaneous, nasal or pulmonary.

According to another mode of realisation of this fourth aspect, the purpose of the invention is a method of early treatment and/or prevention of early relapse of flare-ups of granulomatosis with polyangiitis in a patient suffering from granulomatosis with polyangiitis comprising the administration at an effective dose of at least:
a monoclonal antibody (recombinant and/or isolated and purified) as described above; and/or
a fragment as described above; and/or
a nucleic acid as described above; and/or
an expression vector as described above.

In addition and according to another aspect of the invention, the subject matter of the invention is an antibody/antigen immune complex, in which:
said antibody is the monoclonal antibody (recombinant and/or isolated and purified) as described above or a fragment as described above; and
the antigen is neutrophil proteinase 3 represented by the sequence SEQ ID NO: 1.

The invention also relates to a diagnostic kit for the determination of the blood concentration of neutrophil proteinase 3, said diagnostic kit comprising a monoclonal antibody (recombinant and/or isolated and purified) as described above or a fragment as described above.

On the basis of this aspect, the invention also relates to an in vitro diagnostic method comprising a step of determining the antibody/antigen immune complex as described above using a monoclonal antibody (recombinant and/or isolated and purified) as described above or a fragment as described above,

the said dosage making it possible to determine the degree of severity and/or the risk of relapse of granulomatosis relapses with polyangiitis in a patient.

It has been reported in the literature that the BVAS score (i.e. the score used to quantify the severity of the disease) is correlated with the degree of intensity of membrane expression in PR3. This means that the higher the level of membrane expression in PR3, the greater the severity of the disease and a fortiori the more severe the clinical damage. Since the antibody of the invention as described above specifically targets PR3, the study of the level of membrane expression (fluorescence measurement, MFI) and the percentage of cells expressing PR3 at the membrane through it is therefore an advantageous diagnostic tool. Furthermore, although during the course of the disease the percentage of PR3-positive cells remains unchanged, it appears that neutrophils from patients in the active phase of the disease express more PR3 at the membrane than during the quiescent phase of the disease. Therefore, if this percentage of PR3 membrane expression increases during regular patient monitoring, which can be demonstrated by the antibody of the invention as described above (e.g. labelling of PR3 by flow cytometry), the risk of relapse is quickly identified. The treatment to be adopted can then be quickly and effectively implemented using the correlation between the level of membrane PR3 expression and the severity of GPA.

The present invention is further illustrated, without limitation, by the following figures and examples.

### LIST OF FIGURES

FIG. 1: Identification and characterisation of the human monoclonal anti-proteinase 3 (PR3) 4C3 antibody.

A. Recognition of PR3 by 3 antibodies derived from immunization n° IM3-16 (4C3, 4C5 and 5D11) in ELISA. B. Verification of the specificity of 4C3 and 5D11 in response to different antigens indicated on the graph by ELISA (n=5). C. Determination of the IgG subclass of 4C3 by ELISA in comparison with a GPA (ANCA) patient serum containing different anti-PR3 IgG subclasses (n=3). D. Determination of the kappa (black) or lambda (white) light chain of 4C3 in comparison with a GPA patient serum (n=3). E. Determination of the equilibrium dissociation constant (K_{D}) of the 4C3 by BIACORE™ with respect to the PR3.

FIG. 2: Production and purification of 4C3.

A. Confirmation of the monoclonality of 4C3 by Polymerase Chain Reaction (PCR) by studying the rearrangements of immunoglobulin heavy chain (IgH) genes. B. Production of the 4C3 clone in the supernatants of high-density flasks. (PRO 01-17 and PRO 02-18) and evolution of the number of cells harvested during PRO 01-17 (left graph) and PRO 02-18 (right graph). C. Evaluation of the level of anti-PR3 IgG production by ELISA during PRO 01-17 (left graph) and PRO 02-18 (right graph). D. Purification of 4C3 by affinity chromatography (HiTrap™ Protein A). The purity of the different fractions was controlled by SDS-PAGE and Coomassie Blue staining. NR: Not retained; E: Elutions; Mq: Size marker; Avt: Sample before purification; CTL: Purified IgG1 Ac. E. Recognition of PR3 by purified 4C3 from PRO 02-18 production by anti-PR3 ELISA (coating with 1 μg/mL of PR3).

FIG. 3: Specificity of the recognition of PR3 by the recombinant form of 4C3 (r4C3).

A. Comparison of the binding of 4C3 (black histograms) and recombinant 4C3 (r4C3) (dark grey histograms) to PR3 compared to BSA and ovalbumin (Ova). A representative experiment of the 3 performed is shown. B. Determination of the equilibrium dissociation constant (K_{D}) of r4C3 by BIACORE™ with respect to PR3.

FIG. 4: Modelling and validation of the PR3 epitope recognised by 4C3.

A. Recognition of PR3 by 4C3 by western blot: PR3 in different concentrations and in native or denatured form was deposited on 4-12% Bis-Tris polyacrylamide gel and then transferred to a nitrocellulose membrane and saturated with 5% BSA. Incubation of the membrane with the 4C3 antibody diluted to 1/10,000th one night at 4° C. under agitation. Revelation of the proteins with a secondary anti-human Fc antibody coupled to HRP. B. In silico modelling of the epitope of PR3 recognised by 4C3 by the MabTope method. C. Amino acid sequence of PR3 (SEQ ID NO: 42) with the tested validation peptides underlined. The probability of different residues of the epitope is indicated. D. HTRF measurement of the binding between an anti-Fab d2-coupled antibody that binds to 4C3 and a terbium-coupled streptavidin that binds to the different biotinylated peptides of PR3 (shown on the abscissa). Results expressed as a ratio. ns: not significant. E Amino acid sequence of PR3 whose signal sequence, di-propeptide, pro-peptide, hydrophobic patch, catalytic triad and peptides 3.2 and 4.1 are indicated:
Bold: signal sequence;
AE underlined: di-propeptide cleaved by cathepsin C;
From IVGGH . . . IRSTLR: N-terminal sequence of the active native PR3;
In italics: pro-peptide in C-terminal;
In bold italics: hydrophobic patch;
Bold underlined: catalytic triad;

(SEQ ID NO: 40)
-T [symbol] CRPHNICTFVPR: peptide 4C3_3.2;
and
(SEQ ID NO: 41)
-GTQCLAMGWGRVGAH: peptide 4C3_4.1.

F. Effect of alpha 1 antitrypsin (α1AT) in the binding of 4C3 antibody to PR3 in ELISA. PR3 (2 μg/mL) was incubated or not with alpha 1 anti-trypsin at a ratio of 1 PR3 to 5 α1AT for one hour at 37° C. before being coated in wells overnight at 4° C. The results are expressed as an average±standard deviation of the optical density obtained. *p<0.05. The average of the percentage decrease obtained over the 8 experiments is indicated.

FIG. 5: 4C3 does not inhibit the enzymatic activity of PR3.

PR3 (10 nM) has been incubated or not with 4C3, 6H4 or α1AT (50 nM) during 30 minutes, i.e. a ratio of 5:1, then the PR3 fluorescent substrate was added. Enzyme activity was measured during kinetics by spectrofluorimetry. The negative control (ctl-) corresponds to the substrate alone. A. Representation of the fluorescence obtained at the ratio of 5 Ac 4C3 for 1 PR3. Similar results were obtained with the ratios 10:1 and 2:1. B. Representation of the difference in activity of PR3 expressed in ΔRFU. A representative experiment of 5 is shown.

FIG. 6: Marking of 4C3 on the surface of immune cells in flow cytometry.

A. Analysis strategy for human neutrophils purified by flow cytometry (FACS) after elimination of duplicates (“single cells”) then labelling of the membrane PR3 with the 4C3 antibody coupled with AF488 (graph at bottom right). B. Human neutrophils were purified from the blood of a healthy donor and then incubated or not (histogram “0 μg/ml”) with different concentrations of 4C3 antibodies coupled to AF488 (1, 20 or 100 μg/mL) for 20 minutes at 4° C. in order to study the membrane expression of 4C3. C. Human neutrophils from healthy donors were pre-activated (TNFα+) or not (TNFα−) with TNFα at 2 ng/mL for 15 minutes before being labelled with 4C3-AF488 at 20 μg/ml for 20 minutes. A representative experiment of the 10 carried out is shown. D. Blood cells from a healthy donor were labelled with a CD45-APC mix. H7/CD3-BV786/CD14-VioBlue/CD15-PE/4C3-AF488 before switching to FACS. Representation of PR3 marking with 4C3-AF488 on the surface of T lymphocytes (CD3⁺, light grey histogram), monocytes (CD14⁺, dark grey histogram) and neutrophils (CD15+, hatched histogram). A representative experiment of the 5 performed is shown.

FIG. 7: Cytoplasmic marking of 4C3-AF488 cANCA.

Purified neutrophils from healthy donors were fixed with ethanol (top row) or formalin (bottom row) before being labelled with the nuclear marker DAPI (1^{st} column) and 4C3-AF488 ( 1/100th) (2^{nd} column). Reading the slides with a fluorescence microscope using the 60× objective. Superimpose the fluorescence (“Merged”, 3^{rd} column) using the ImageJ software. The scale (10 μm) is indicated on the phase contrast images (“Brightfield”, 4th column) A representative experiment of the 3 experiments is shown.

FIG. 8: 4C3 specifically recognises purified native PR3 and PR3 contained in neutrophils.

A. Neutrophils from a GPA patient were purified and then pre-activated (TNFα+) or not (TNFα−) with TNFα at 2 ng/mL for 15 minutes at 37° C. 10 μg of neutrophils (wells 1 and 2) were deposited in a reduced condition. HeLa cells without PR3 were used as a negative control (3^{rd} well) and purified native human PR3 as a positive control (4^{th} well). Incubation of the membranes with Hsc70 antibody ( 1/10,000^{th}) or 4C3 antibody ( 1/10,000^{th}) overnight under agitation at 4° C. B. The 4C3 does not bind the proteases Elastase and Cathepsin G (Cath G) but only PR3 at 5 μg and 2 μg. Incubation of 4C3 overnight at 4° C.

FIG. 9: 4C3 induces the expression of PR3 on the surface of pre-activated neutrophils against a non-relevant antibody.

Neutrophils from healthy donors were pre-activated by TNFα at 2 ng/mL at 37° C. for 15 minutes (triangle) and then incubated for 90 minutes at room temperature with monoclonal antibody (MAb) 4C3 (circle) and MAb 6H4 (square) at 2 μg/mL (n=3), at 20 μg/mL (n=4) and at 100 μg/mL (n=2) or with PMA-ICa (inverted triangle). The membrane expression of PR3 (mbPR3) was analysed by flow cytometry after labelling with the anti-PR3 antibody WGM2-FITC. The mean as well as standard deviations for healthy donors are shown. The results have been normalised to the TNFα condition and are expressed as a ratio of Mean Fluorescence Intensity (MFI). *p<0.05.

FIG. 10: 4C3 does not induce the production of reactive oxygen species (Reactive Oxygen Species; ROS) by human neutrophils.

Purified neutrophils from eight independent healthy donors were pre-activated with TNFα (2 ng/mL) for 15 minutes at 37° C. (white columns) before being incubated for 45 minutes with 4C3 (grey columns), IgG preparations (unmixed) from two healthy donors (hatched columns) or from four GPA patients active at the time of diagnosis (gridded columns) or IgG from patient P2 (vertical rows). The production of ROS was evaluated by measuring the fluorescence (MFI) of DHR 123 by flow cytometry. The results are expressed as an average±SEM obtained in eight independent experiments, each circle representing one experiment. NS: not significant; *p<0.05; **p<0.005; ***p<0.0005.

FIG. 11: Dose effect of 4C3 on the intracellular production of reactive oxygen species (Reactive Oxygen Species; ROS) by neutrophils.

Neutrophils from healthy donors were incubated with cytochelin B (5 μg/mL). 5 minutes at 37° C. then sodium azide (2 mM) and DHR (2.5 μM) were added. 5 minutes at 37° C. before pre-activating neutrophils with TNFα at 2 ng/mL at 37° C. for 15 minutes (triangle). At the end of the pre-activation, the neutrophils were incubated 45 minutes at 37° C. with 4C3 AcMo (circle) or 6H4 AcMo (square) at 2 μg/ml (n=9), at 20 μg/mL (n=20) and at 100 μg/mL (n=4) or with PMA-ICa (inverted triangle). The production of ROS was analysed by flow cytometry by measuring the MFI after marking with DHR 123. The mean as well as standard deviations for healthy donors are shown. The results have been normalised to the condition TNFα and are expressed as a ratio of MFI. ns: not significant; *p<0.05; **p<0.005.

FIG. 12: 4C3 and r4C3 do not induce an increase in cathepsin G activity by pre-activated neutrophils.

A. Purified neutrophils from eight independent healthy donors were pre-activated with TNFα (2 ng/mL) for 15 minutes at 37° C. (white columns) before being incubated for 45 minutes with 4C3 (grey columns), IgG preparations (unmixed) from two healthy donors (hatched columns) or from four GPA patients active at the time of diagnosis (gridded columns) or IgG from patient P2 (vertical rows). Neutrophil degranulation was assessed by CD63 expression as a percentage of positive cells. The results are expressed as an average±SEM obtained in eight independent experiments, each circle representing one experiment. NS: not significant; *p<0.05; **p<0.005; ***p<0.0005. B. Neutrophils from healthy donors were pre-activated by TNFα at 2 ng/mL at 37° C. for 15 minutes (left histogram) and then incubated for 90 minutes at room temperature with 2 and 20 μg/mL r4C3 and r4C3 MAc or with PMA-ICa. Cathepsin G activity was measured in neutrophil activation supernatants after addition of its substrate and reading by spectrofluorimetry. A representative experiment of 3 is shown. The results are expressed in ΔRFU (Relative Fluorescence Units).

FIG. 13: 4C3 does not increase the adhesion phenotype of human neutrophils.

A and B. Purified neutrophils from eight independent healthy donors were primed with TNFα (2 ng/mL) for 15 minutes at 37° C. (white columns) before being incubated for 45 minutes with 4C3 (grey columns), separate IgG preparations (unmixed) from two healthy donors (hatched columns) or from four GPA patients active at the time of diagnosis (gridded columns) or IgG from patient P2 (vertical rows). The neutrophil adhesion criterion was evaluated by measuring the surface expressions of CD11b (A) and CD18 (B) by flow cytometry. The results are expressed as an average±SEM obtained in eight independent experiments, each circle representing one experiment. NS: not significant; *p<0.05; **p<0.005; ***p<0.0005.

FIG. 14: Analysis by SDS-PAGE of the different shapes of 4C3 obtained.

4C3 native (well 2), 4C3 recombinant (well 3), 4C3 deglycosylated (well 5) and 4C3 Fab (well 6) compared to 6H4 (well 4; anti-ovalbumin IgG1). The left well (No. 1) corresponds to the molecular weight (MW) marker in kilo Dalton (kDa).

FIG. 15: Derivatives of 4C3 do not increase PR3 expression and do not induce the production of SAR by pre-activated neutrophils.

A. Neutrophils from healthy donors were pre-activated by TNFα at 2 ng/mL at 37° C. for 15 minutes and then incubated for 90 minutes at room temperature with MoA 4C3 and MoA 6H4 at 40 μg/mL or with PMA-ICa or in mol equivalent for Fab and F(ab′)₂. The membrane expression of PR3 (mbPR3) was analysed by flow cytometry after labelling with 4C3-AF488 antibody. An experiment representative of 3 realised is shown. B. The production of ROS was measured in kinetics in the presence of the Fab, F(ab′)₂ and deglycosylated Fc (dFc) derivatives of the 4C3 antibody. n=1.

FIG. 16: r4C3 does not increase the intracellular production of SAR by neutrophils. Neutrophils from healthy donors were pre-activated with TNFα (triangle) and then incubated for 90 minutes with r4C3 (circle, n=11), 4C3 (diamond, n=20) or with PMA-ICa (inverted triangle). The production of ROS was analysed by flow cytometry by measuring the MFI after marking with DHR 123. The mean as well as standard deviations for healthy donors are shown. The results have been normalised to the condition TNFα and are expressed as a ratio of MFI. NS: not significant.

FIG. 17: Analysis in SDS electrophoresis—PAGE of the different fractions obtained after purification of a GPA patient serum on G protein.

Serum (well 2), unbound fraction (NR, well 3), washes (L, wells 4 to 9), elutions (E, wells 10 to 14). The left (No. 1) and right (No. 15) wells correspond to the molecular weight (MW) marker in kDa. Representative image of the 6 experiments carried out.

FIG. 18: Purified IgG from GPA patient serum contains anti-PR3 IgG in contrast to purified IgG from healthy donor serum.

The IgG contained in the serum of healthy donors and GPA patients was purified and then the anti-PR3 IgG assay was performed by ELISA using the Eurolmmun™ kit. The kit's internal controls (negative control/positive control/20 UR/mL calibrator) were used in comparison with the 50 μg/mL AcMo 4C3 and unpurified sera. A representative experience of the 6 performed is shown.

FIG. 19: Purified IgG induces an increase in the intracellular production of ROS by neutrophils from healthy donors.

Neutrophils from healthy donors were pre-activated with TNFα at 2 ng/mL at 37° C. for 15 minutes (triangle) and then incubated with purified IgG from GPA patients (circle, n=32) or from healthy donors (diamond, n=16) at 200 μg/mL or with 4C3 (inverted triangle, n=20). The production of ROS was analysed by flow cytometry by measuring the MFI after marking with DHR 123. The mean as well as standard deviations are shown. The results have been normalised to the condition TNFα and are expressed as a ratio of MFI. ns: not significant; *p<0.05; **p<0.005; ***p<0.0005.

FIG. 20: 4C3 is capable of inhibiting the intracellular ROS production of human neutrophils induced by the presence of IgG GPA+.

Neutrophils from healthy donors were incubated with cytochelin B (5 μg/mL). 5 minutes at 37° C. then sodium azide (2 mM) and DHR (2.5 μM) were added. 5 minutes at 37° C. before pre-activating the neutrophils with TNFα at 2 ng/mL at 37° C. for 15 minutes. At the end of pre-activation, neutrophils were incubated for 15 minutes at 37° C. with AcMo 4C3 at 20 μg/mL before adding IgG from patients with GPA at 200 μg/mL for 35 minutes at 37° C. ROS production was analysed by flow cytometry by measuring MFI after labelling with DHR 123. The results obtained with 4 IgG from GPA patients are shown. The results are expressed in MFI and the percentage of inhibition induced by 4C3 is shown. n=4.

### EXAMPLES/MATERIALS & METHODS/RESULTS

1. Patient Selection

Before the 4C3 antibody was obtained, three immortalisations were performed from blood samples of 3 different patients with Wegener's disease or Granulomatosis With Polyangiitis (GPA). Clones could be obtained but none of them resulted in the production of a relevant antibody. The causes of these failures being multiple: lymphopenia, thrombocytosis, hyperleukocytosis, it was decided to adapt the process by targeting patients more specifically according to the clinic and biology, and by optimising the immortalisation technique.

The 4C3 clone is derived from a patient suffering from granulomatosis with polyangiitis (GPA) who was followed up in consultation in the pneumology department of the Tours Regional and University Hospital Centre (CHRU). This patient was diagnosed with GPA in 2011 following ear, nose and throat (ENT) and lung problems and an alveolar haemorrhage. This patient received an initial 6-month treatment with Endoxan™ followed by corticosteroids and methotrexate. The last biological examination at the time of sampling indicated a circulating B-cell count of 72/mm³ and a of autoantibodies against proteinase 3 (cANCA) greater than 177 IU/mL. The patient agreed to participate, after informed consent, in a collection of human biological samples declared to the Ministry of Research (n°DC-2012-1636) in accordance with decree n°2007-1120 of 10 Aug. 2007. After collecting clinical data and signing the informed consent, the patient's blood was collected on ACD (Dextrose Citrate anti-coagulant) and then sent via the immunology laboratory of the Tours CHRU to the BCRessources platform.

2. Immortalisation of B-Lymphocytes

To obtain anti-PR3 IgG-producing clones (experiment n°IM3-16), a kit marketed by the company Dendritics™ (DDXK-HuBB™ kit) was used, which uses the EBV (Epstein Barr Virus) immortalisation technique. Nevertheless, the protocol recommended by this company has been optimised by the inventors and is different from the one described in the kit. Normally, the kit is used on fresh total Peripheral Blood Mononuclear Cells (PBMC) (from a daily blood sample) and requires a first immortalisation step and then sub-cloning steps to obtain monoclonal cells. To obtain the 4C3 antibody, optimisation of the kit led to the use of thawed (and not fresh) PBMCs, which were then enriched by cell sorting with autologous memory B lymphocytes (LB) in the culture wells, i.e. a new stage of the protocol was developed by the inventors. In addition, and as a consequence of this optimisation, the culture conditions that were put in place also made it possible to dispense with the subcloning step, which is described as essential in the kit protocol.

More precisely, for the enrichment in memory B lymphocytes, two successive steps were carried out: the first step allowed the isolation by negative selection of the total B lymphocytes (“B-cell isolation kit II” from Miltenyi Biotec˜, Ref 130-091-151) and then the specific sorting of the CD19⁺ CD27⁺ memory B lymphocytes with the Astrios MoFlo™ sorter. Different co-culture conditions were tested in flat-bottom P96: PBMC alone, PBMC enriched in memory B-lymphocytes, PBMC depleted in memory B-lymphocytes and enriched in memory B-lymphocytes with different quantities of cells During the second step, the cells were stimulated and immortalised in the presence of EBV viral particles using the kit marketed by Dendritics™ (kit DDXK-HuBB™). During the first ten days of culture, cluster formation was evaluated under the microscope and the cells were restimulated after 9 days (D9) of culture.

3. Selection of Clones by ELISA

From culture days 20, the identification of IgG producing clones by ELISA has been started. Total IgG positive supernatants were identified and the corresponding clone cultures were continued. Conversely, wells that came out twice negative were eliminated. In a first step, 62 clones were selected on the basis of their detectable total IgG production.

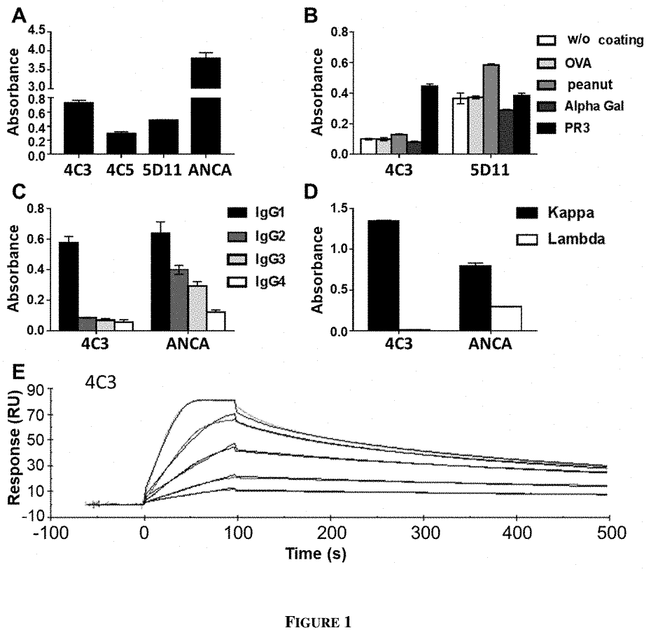

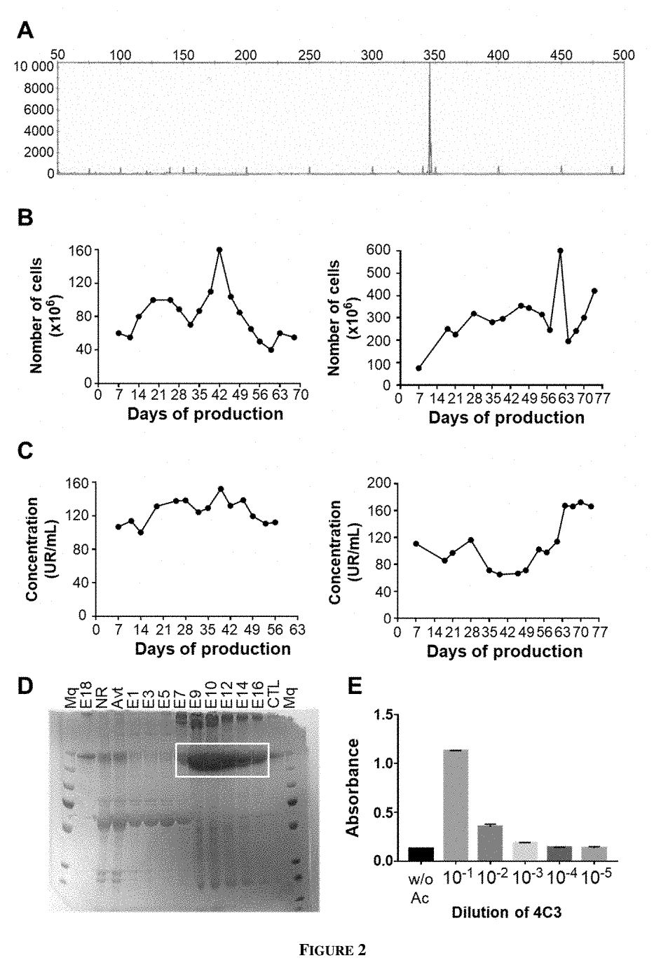

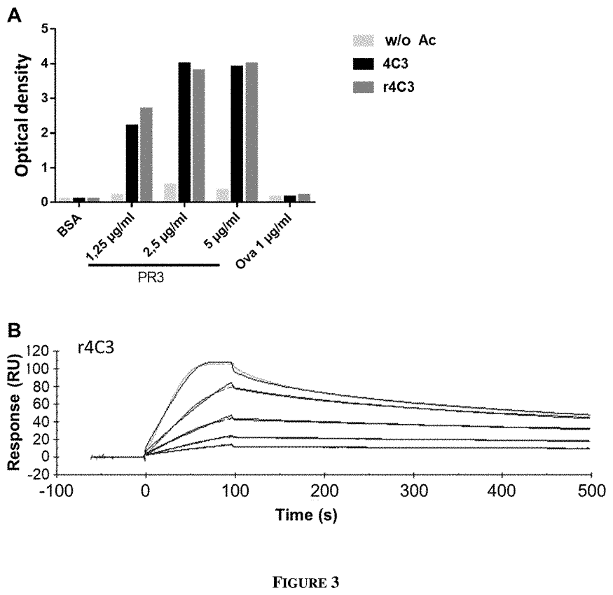

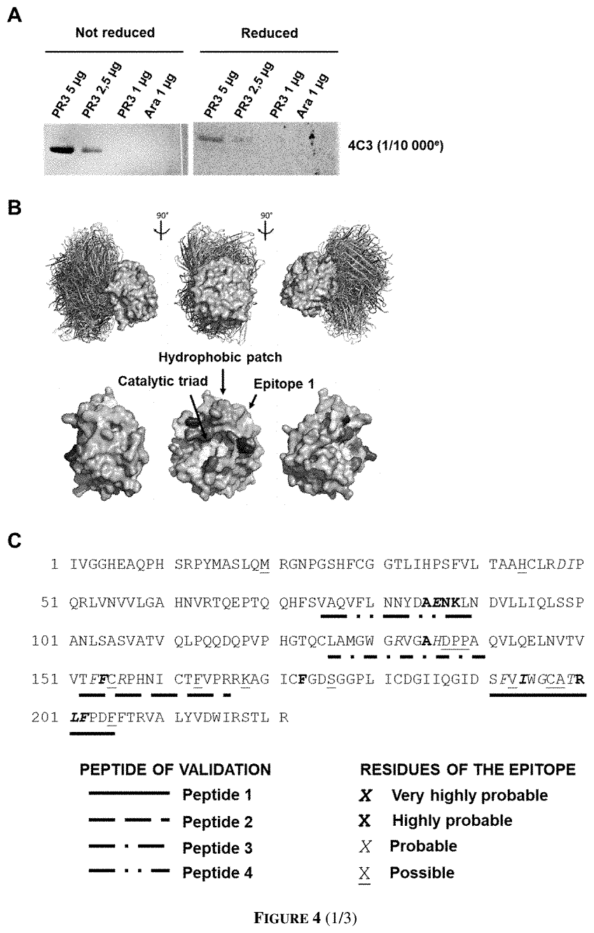

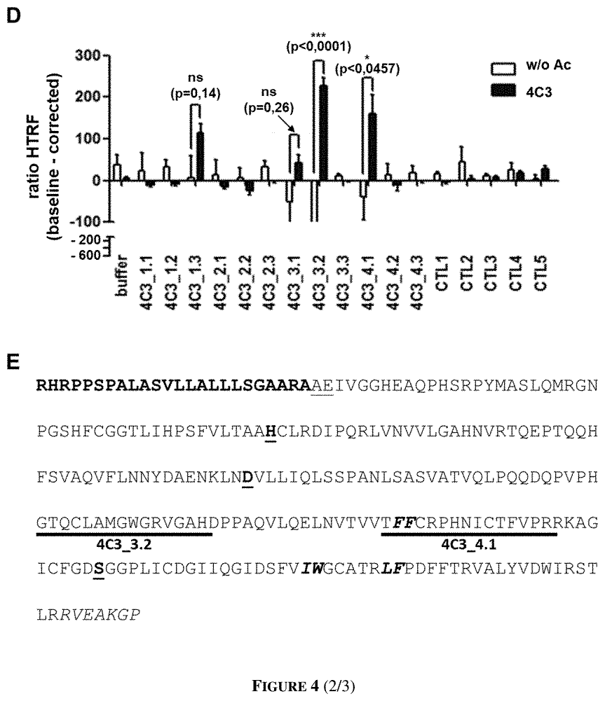

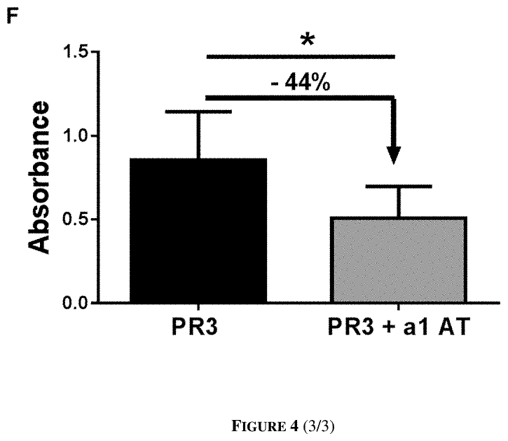

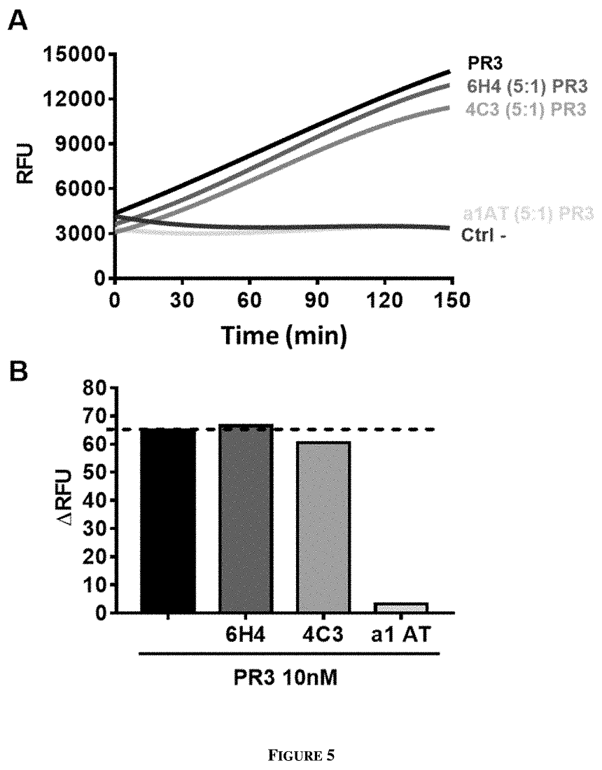

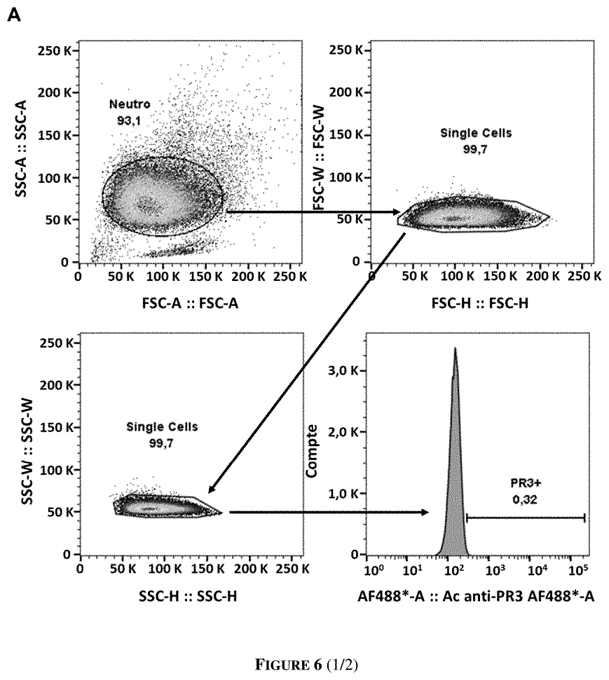

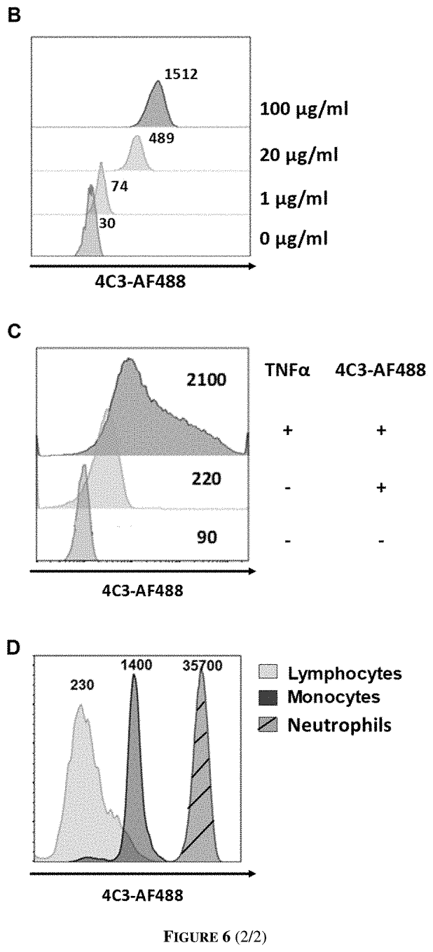

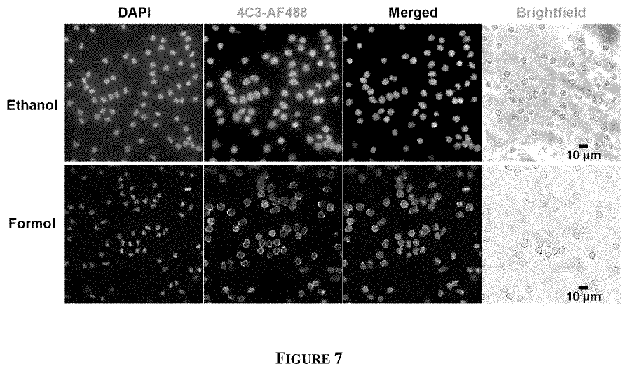

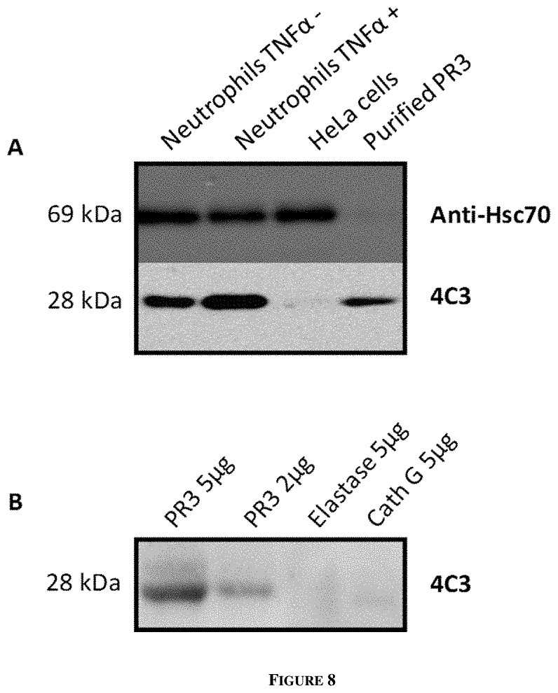

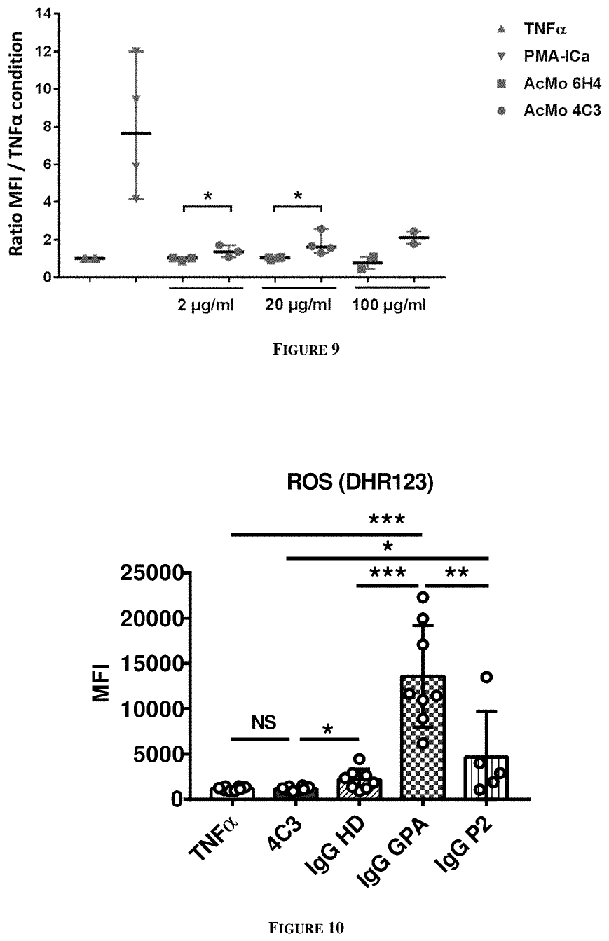

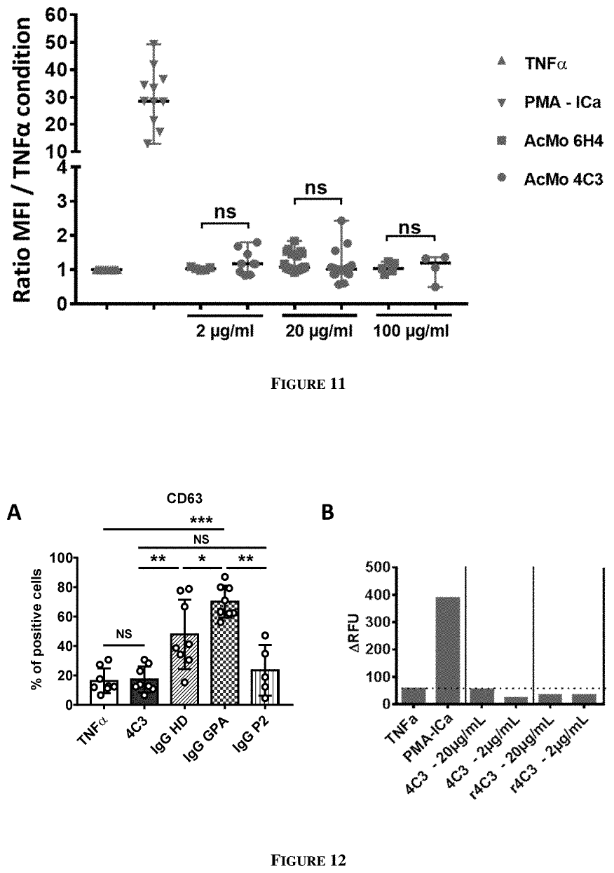

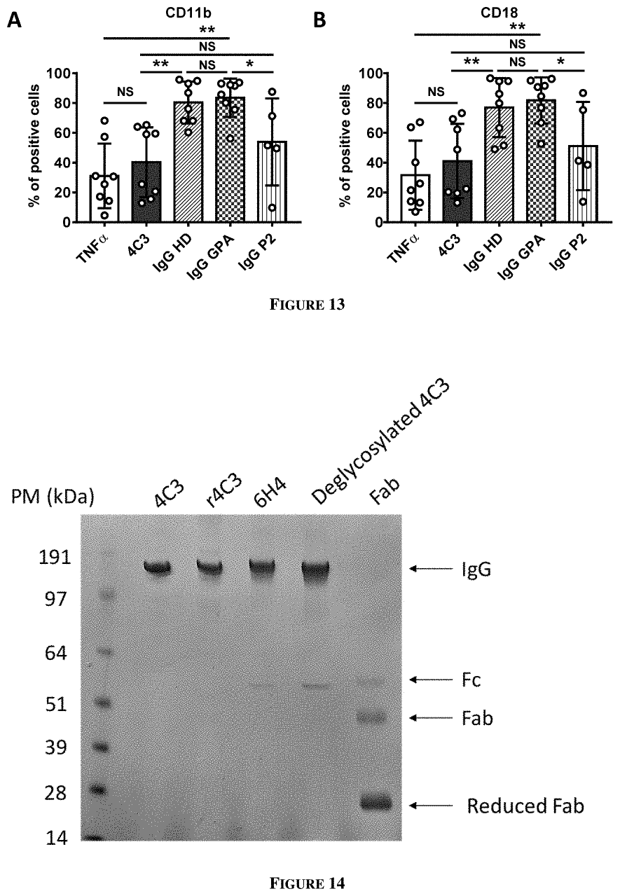

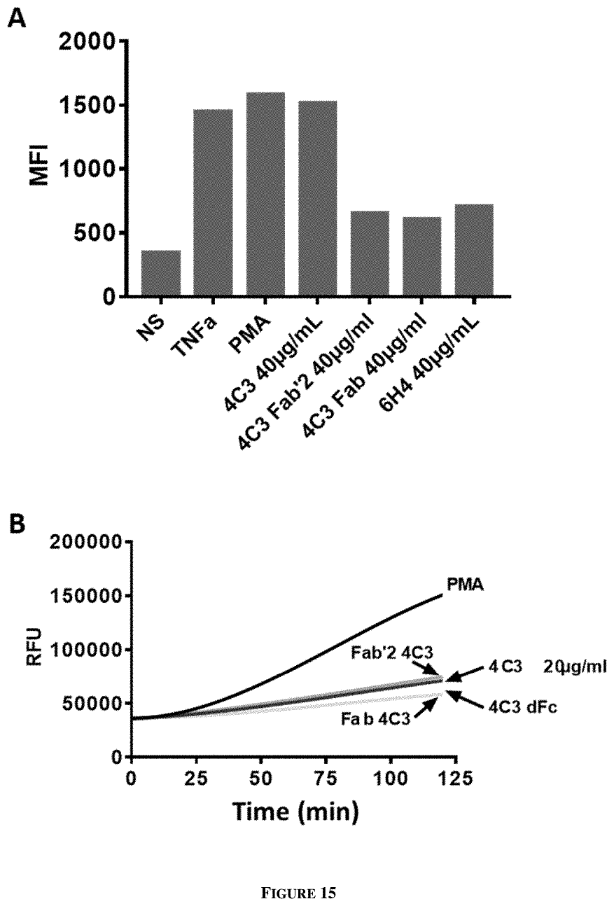

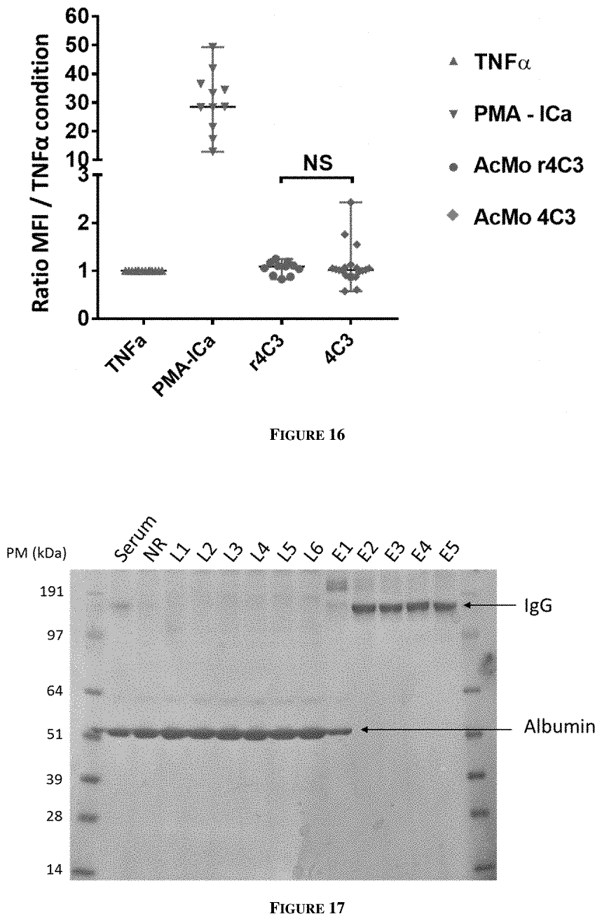

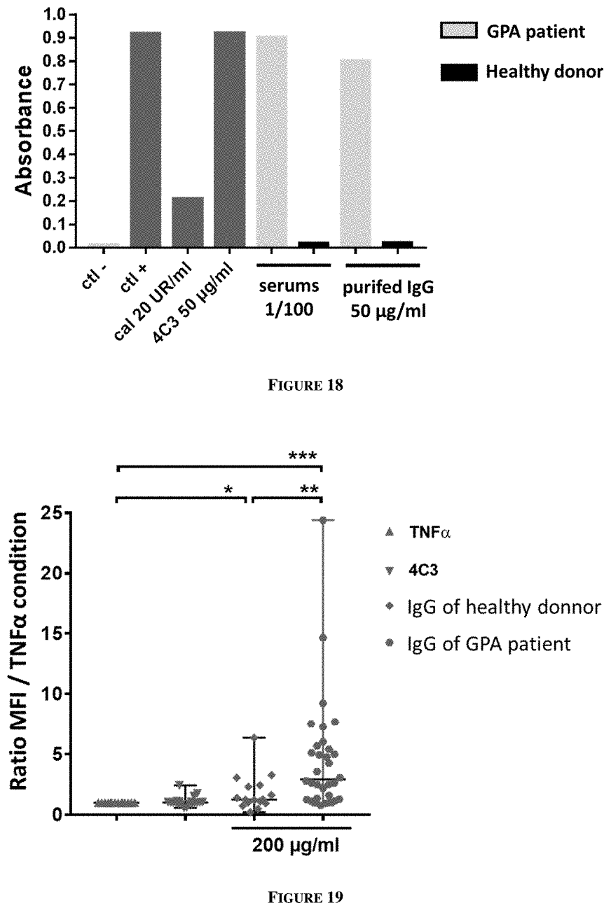

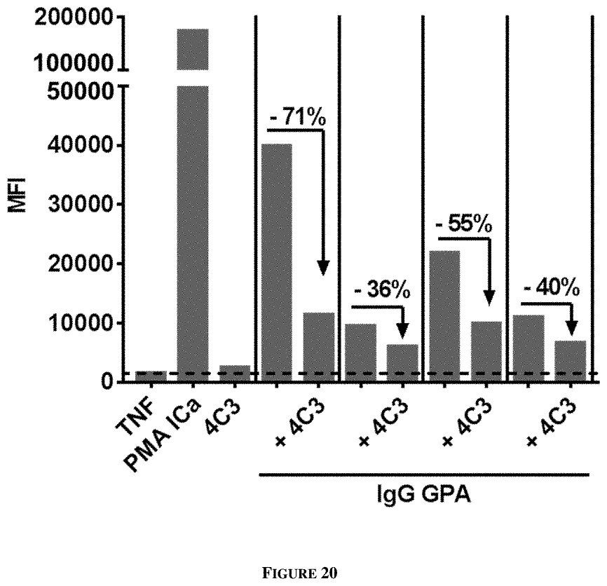

These positive clones were again screened by ELISA in the presence of PR3 (Athens Research and Technology™, reference 16-14-161820). After coating the wells of the MaxiSorp™ ELISA plate (96 wells) with 2 μg/mL of native PR3 overnight at 4° C., the wells were saturated with 4% PBS-BSA (Phosphate Buffer Saline—Bovine Serum Albumin) buffer to prevent non-specific binding of antibodies to the well plastic. The B-cell culture supernatants were incubated for 2 hours at 37° C. before adding the secondary antibody, i.e. anti-Fc human IgG coupled with HRP (HorseRadish Peroxidase). The revelation was made in the presence of TMB (3,3′,5,5′-TetraMethylbenzidine), peroxidase substrate. The absorbance reading was taken at a wavelength of 620 nm. The threshold of positivity was set at twice the absorbance value of the negative control. Cells whose supernatants came out positive at least three times in ELISA were amplified in order to have enough cells for further testing. Conversely, wells that tested negative twice in a row were discarded. Thus, only 5 clones tested positive in anti-PR3 ELISA. The 5 clones were expanded, re-tested by ELISA and the best 3 were retained (i.e. 4C3, 4C5 and 5D11) (FIG. 1A). Only 4C3 was found to be specific for PR3, 5D11 being non-specific (FIG. 1B) and 4C5 having stopped producing IgG.

4. Characterisation of 4C3 Antibody

After confirmation of the specificity of the 4C3 antibody (FIG. 1B), it was shown by ELISA (commercially available ELISA tests) that this anti-PR3 antibody of the IgG isotype had an IgG1 subclass and a kappa chain (FIGS. 1C and 1D). The affinity of 4C3 for PR3 is high with a dissociation constant K_{D} of 7.41.10-10 M (value obtained by the BIACORE technique) (FIG. 1E+Table 2 below).

TABLE 2
Affinity of 4C3 antibody
4C3

| | ka (1/Ms) | kd (1/s) | K_{D} (M) | Rmax | | Chi² (RU²) | U-value |
|---|---|---|---|---|---|---|---|
| Curve | 1.26E+07 | 0.00936 | 7.41E−10 | (RU) | Conc (M) | 0.723 | 3 |

| | | |
|---|---|---|
| Cycle: 5 | 78.35 | 1.25E−09 |
| Cycle: 6 | 76.5 | 2.50E−09 |
| Cycle: 7 | 80.18 | 5.00E−09 |
| Cycle: 8 | 71.98 | 1.00E−08 |
| Cycle: 9 | 75.01 | 2.00E−08 |

5. Production in High Density Flasks

After an initial characterisation of the antibody secreted in the culture supernatants, clone 4C3 was amplified by successive passages of the cells in P24 wells, then in P6 wells and finally in flasks in order to increase the number of cells. The monoclonality of this clone was confirmed by Polymerase Chain Reaction (PCR) by studying the rearrangements of immunoglobulin heavy chain (IgH) genes (FIG. 2A). The 4C3 clone was then put into production in a high-density flask (CELLine™ Wheaton™) composed of two compartments separated by a semi-permeable membrane that allows the passage of proteins below 10 kDa. The first compartment contains the extracellular nutrient medium composed of DMEM plus penicillin/streptomycin and glutamine but without horse serum while the second compartment contains the cells in which the antibodies produced every three to four days over a period of at least 70 days were collected.

Two productions were carried out (experiments PRO 01-17 and PRO 02-18) during which the number of cells (FIG. 2B) and the level of anti-PR3 IgG production (FIG. 2C) were checked.

6. Purification of 4C3 Antibody

All the supernatants collected were filtered through a 0.2 μm filter after centrifugation. 10 minutes at 500 g. The antibodies contained in the supernatants were purified by affinity chromatography (HiTrap™ Protein A 1 mL GE Healthcare™) on the AKTA™ (GE Healthcare™) apparatus. The purity of the different fractions was controlled by 4-12% Bis-Tris gel electrophoresis and Coomassie Blue staining (FIG. 2D). The final concentration of 4C3 antibody, after BCA™ assay, is 5,974 mg/mL. The specificity of 4C3 was confirmed by ELISA in the presence of coated native PR3 (1 μg) (FIG. 2E).

7. Specificity of 4C3 in its Recombinant Form

In order to produce the recombinant 4C3 antibody (r4C3), the sequences of the PR3-specific variable regions (SEQ ID NOs: 6 and 24) were subcloned into recombinant IgG expression vectors (commercially available vectors). The expression vectors thus constructed were transfected into HEK-293 mammalian cells (ATCC® CRL-1573™) for the production of monoclonal IgG isotype in the form secreted in the culture supernatant. After harvesting 50 mL of culture supernatant, the r4C3 antibody was purified as previously described and yielded a final r4C3 concentration of 3.5 mg/mL.

To confirm the specificity of the recombinant 4C3 antibody, an ELISA test was performed in the presence of different amounts of coated PR3 compared to 4C3 (FIG. 3A). It was shown that r4C3 is able to bind to PR3 in a manner comparable to 4C3 (FIG. 3A). This result has been confirmed by BIACORE as r4C3 has an affinity to PR3 comparable to 4C3 (FIG. 3B+Table 3 below).

TABLE 3
Affinity of the r4C3 antibody
r4C3

| | ka (1/Ms) | kd (1/s) | K_{D} (M) | Rmax | | Chi² (RU²) | U-value |
|---|---|---|---|---|---|---|---|
| Curve | 1.37E+07 | 0.009358 | 6.84E−10 | (RU) | Conc (M) | 1.06 | 4 |

| | | |
|---|---|---|
| Cycle: 14 | 161.8 | 1.25E−09 |
| Cycle: 15 | 128.1 | 2.50E−09 |
| Cycle: 16 | 110.9 | 5.00E−09 |
| Cycle: 17 | 98.36 | 1.00E−08 |
| Cycle: 18 | 100.5 | 2.00E−08 |

The two 4C3 and r4C3 antibodies share the same variable region of the heavy chain (SEQ ID NOs: 6 to 19) and the same light chain (SEQ ID NOs: 20 to 35) but differ only in the constant region of the heavy chain by the mutation R>K in position 224 (or AGA>AAA in position 670-672). This mutation confers a different allotype to these 2 antibodies, namely G1m3-1 for 4C3 and G1m17-1 for r4C3.

8. Identification of the Epitope Recognised by 4C3

In order to identify the epitope of PR3 recognised by 4C3, a western blot in a non-denaturing and denaturing condition was first made. Proteinase 3 (native or denatured) was separated by electrophoresis on 4-12% Bis-Tris polyacrylamide gel, then transferred to a nitrocellulose membrane and saturated with 5% BSA in 0.1% Tween™ 20 TBS at room temperature for one hour under agitation. The membrane was then incubated with 4C3 ( 1/10,000^{th} dilution) overnight at 4° C. under agitation. To reveal the proteins, the membranes were incubated with a secondary anti-human Fc antibody coupled to HRP and revealed with the Pierce™ ECL Plus (ThermoFisher™) kit. It was thus determined that 4C3 preferentially recognised a conformational epitope of PR3 with observation of a less intense labelling of PR3 in denaturing condition (FIG. 4A).

The identification of the epitope recognised by the 4C3 antibody was carried out by MabSilico. The first step enabled the PR3 epitope to be modelled using the MabTope method, which consists of the computer prediction of an ordered list of PR3 peptides likely to belong to the epitope from the sequence of the variable part (V_{H} and V_{L}) of 4C3 (FIGS. 4B and 4C). The second step allowed the experimental measurement of the specific binding of a number of these peptides to the antibody. This measurement was made by HTRF (Homogeneous Time Resolved Fluorescence) between an anti-Fab d2-coupled antibody (CisBio™) which binds to the antibody of interest, and a terbium-coupled streptavidin (CisBio™) which binds to the biotinylated peptide (FIG. 4D). The peptide 4C3_3.2 (sequence SEQ ID NO: 40) induced the highest HTRF ratio (FIG. 4D) thus demonstrating that the epitope recognised by the 4C3 consists of a majority of these amino acids (FIG. 4E).

In addition, the binding of 4C3 to PR3 was altered in the presence of alpha 1 anti-trypsin (α1AT), the natural PR3 inhibitor, which is able to induce a conformational change in PR3 that prevents anti-PR3 Ac's that recognise epitope 1 from binding to it (FIG. 4F).

4C3 therefore targets a conformational epitope of PR3 close to its catalytic site (amino acids in bold underlined, FIG. 4E) and to the hydrophobic patch (amino acids in italic bold, FIG. 4E) and over a region overlapping that of epitope 1 of PR3 (FIG. 4B).

9. Enzymatic Activity of PR3 in the Presence of 4C3

To better characterise the 4C3 antibody, the enzymatic activity of PR3 was measured after incubation with 4C3 for 30 minutes at different ratios (10 Ac: 1 PR3/5 Ac: 1 PR3 and 2 Ac: 1 PR3) and after addition of the commercial PR3 fluorescent substrate. (Abz-VADnVADYQ-YNO2). It has thus been shown that 4C3 does not inhibit the enzymatic activity of PR3 under these experimental conditions, just like 6H4, which is a non-relevant antibody, whatever the ratio used (FIG. 5). Indeed, in the presence of these antibodies, the substrate was degraded (increased fluorescence) whereas the addition of alpha 1 anti-trypsin (1AT) which is a natural PR3 inhibitor did not induce any degradation of the PR3 substrate (FIG. 5).

10. Labeling of the Membrane PR3 by Flow Cytometry

The ability of the 4C3 antibody to bind to PR3 was also validated by flow cytometry. For this purpose, neutrophils were purified from the blood of healthy donors at the Etablissement Français du Sang (EFS) using a commercially available kit (Stem Cell kit, EasySep™ Direct Human Neutrophil Isolation; Kit reference 19666) (FIG. 6A). Unstimulated neutrophils were incubated for 30 minutes at 4° C. with 4C3 antibody previously coupled with fluorochrome AF488 (kit marketed ThermoFisher™). Several concentrations of 4C3 were tested (0, 1, 20 or 100 μg/mL) and revealed a more intense labelling of PR3 on the surface of the neutrophils depending on the concentration of coupled antibody used (FIG. 6B). In addition, it has previously been described in the literature that priming neutrophils with TNF alpha (TNF) induced an increase in membrane PR3 expression. This was confirmed by an increase in labelling with 4C3-AF488 after stimulation of neutrophils for 15 minutes at 37° C. with TNF alpha (10 ng/mL) demonstrating that Ac 4C3-AF488 recognises PR3 well (FIG. 6C). Finally, this result was reinforced by the absence of 4C3-AF488 labelling on the surface of lymphocytes (lymphocytes not expressing membrane PR3), intermediate labelling on monocytes (PR3 weakly expressed) and very strong labelling on neutrophils (PR3 constitutively expressed) (FIG. 6D).

11. Labeling of Cytoplastic 4C3 cANCA Type

Purified neutrophils from healthy donors were first fixed with formalin or ethanol and then labelled with Ac 4C3 coupled with AF488 and DAPI (nuclear labelling). Analysis of the slides under a fluorescence microscope revealed a diffuse cytoplasmic cANCA-type labelling of 4C3 (FIG. 7).

12. Recognition of PR3 by the 4C3 in Western Blot

To confirm anti-PR3 specificity, 4C3 was used as a primary antibody in western blot (FIGS. 8A and 8B) on protein lysates from neutrophils, HeLa cells (ATCC® CCL-2™) and human PR3. According to FIG. 8, 4C3 did recognise human PR3 with a size-labelling at the expected size, which was also present with neutrophils and no labelling was detected in HeLa cells, which do not express PR3 (FIG. 8A). In addition, 4C3 did not recognise other proteases such as elastase and cathepsin G which are the same size as PR3 (FIG. 8B). This result therefore confirmed the fact that 4C3 antibody is specific for PR3.

13. Depletion of 4C3 Fc Fragments

In order to produce anti-PR3 antibodies that neutralise the ANCA-PR3 interaction and thus decrease neutrophil activation during GPA, different fragments of 4C3 were tested after depletion of the Fc fragments of 4C3, which are capable of activating neutrophils via the Fc/FcγR interaction. To do this, two enzymes were used: pepsin and papain. Pepsin is a proteolytic enzyme that allows the antibody to be cleaved just below the disulphide bridges that link the two heavy chains to obtain an F(ab′)₂ fragment (Table 4 below). Papain is a cysteine protease capable of cleaving the antibody above the hinge region to give two distinct Fab fragments on the one hand and Fc fragments on the other (Table 5 below).

TABLE 4
List of samples deposited by track
DIGESTION OF 4C3 BY PEPSIN

| Track | Samples | mg/mL |
|---|---|---|
| 1 | F(ab′)₂ (Flowthrough Protein A) | 0.259 |
| 2 | F(ab′)₂ (Flowthrough Protein A) | 0.257 |
| 3 | F(ab′)₂ (Protein A wash) | 0.068 |
| 4 | F(ab′)₂ (Protein A wash) | 0.034 |
| 5 | E1 Protein A | 0.051 |
| 6 | E2 Protein A | 0.044 |
| 7 | E2 Protein A | 0.051 |
| 8 | 4C3 purified | 5.9 |

TABLE 5
List of samples deposited by track
DIGESTION OF 4C3 BY PAPAIN

| Track | Samples | mg/mL |
|---|---|---|
| 1 | Fc (E1) | 0.294 |
| 2 | Fc (E2) | 0.231 |
| 3 | Fc (E3) | 0.012 |
| 4 | Fab (Flowthrough Protein A) | 0.542 |
| 5 | Fab (Protein A wash) | 0.166 |
| 6 | E1 2^{nd} pass of Fab on Protein A | 0.002 |
| 7 | E2 2^{nd} pass on Fab on Protein A | 0.0027 |
| 8 | E3 2^{nd} pass on Fab on Protein A | 0.044 |
| 9 | 4C3 purified | 5.9 |

14. Functional Tests on Neutrophils

Incubation of ANCA with neutrophils pre-activated by TNFα leads to degranulation, production of ROS and the formation of NET (Neutrophil Extracellular Traps) and therefore to neutrophil activation. In order to test the neutralising action of 4C3 and its derivatives (r4C3, F(ab′)₂, Fab, deglycosylated 4C3 . . . ) on human neutrophils, various in vitro functional tests have been set up.

Material & Methods

14.1 Purification of IgG from Serum

For functional tests, we have selected sera from patients with active disease according to clinical examinations, treatment and PR3-ANCA level. Patients with an active disease were diagnosed at the University Hospital Centre of Tours. We purified IgG from five sera of independent GPA patients during the active phase of the disease after diagnosis (IgG GPA) (see Table 6 below) or during remission for patient P2 (IgG P2) and two independent healthy donors with a rapid flow Sepharose™ 4 protein G kit (GE HealthCare®, USA). Briefly, unmixed sera were incubated with protein G for one hour at room temperature before elution. The samples were filtered using a Spin-X® UF Concentrator ultrafiltration system. The purified IgG from the unmixed sera was subjected to electrophoresis on a Bis-tris 4-12% before being coloured with Coomassie blue. The presence of PR3-ANCA in separate IgG preparations from active GPA patients and patient P2 was confirmed by ELISA using a EuroImmun anti-PR3 kit, whereas separate purified IgG preparations from healthy donors did not contain PR3-ANCA.

TABLE 6
Main characteristics of active GPA patients whose IgG has
been purified to induce autoimmune neutrophil activation

| | GPA 1 | GPA 2 | GPA 3 | GPA 4 | GPA 5 |
|---|---|---|---|---|---|
| Gender | Male | Male | Male | Male | Male |
| Age at | 68 years | 71 years | 79 years | 77 years | 53 years |
| diagnosis | old | old | old | old | old |
| Reach | Renal, | Joint | Renal | Lung | Renal |
| | neurological | and | and | and | and |
| | and ENT | ENT | joint | ENT | pulmonary |
| Type of | cANCA | cANCA | cANCA | cANCA | cANCA |
| ANCA | | | | | |
| Level of | 83 | 36 | 43 | 112 | 58 |
| PR3-ANCA | IU/mL | IU/mL | IU/mL | IU/mL | IU/mL |

14.2 Neutrophil Purification and Pre-Activation by TNFα

Human neutrophils from healthy independent donors have been purified by negative magnetic selection with the commercial “EasySep® Direct Human Neutrophil Isolation Kit” (StemCells®, Canada) following the manufacturer's instructions. At the end of the isolation, the neutrophils were suspended in a calcium- and magnesium-free HBSS solution. The purity of the isolated neutrophils was >95%, after evaluation by flow cytometry (CD15-PE and Live dead, Miltenyi Biotech®, Germany). The cells were pre-activated with TNFα (Sigma-Aldrich®, USA) at a final concentration of 2 ng/mL for 15 minutes at 37° C. in a water bath.

14.3 Assessment of SAR Production by Neutrophils

Neutrophil activation from healthy independent donors was assessed by the production of ROS using a dihydrorhodamine 123 (DHR 123) test. Purified neutrophils were suspended in HBSS with 1 mM Ca2+ and 1 mM Mg2+ and incubated with 5 μg/mL cytochalasin B (Cayman Chemical®, USA) to increase the production of oxygen radicals, for 5 minutes at 37° C. The cells were then loaded with 2 μM DHR 123 and 2 mM sodium azide (NaN3) for 5 minutes at 37° C. with agitation. Primed neutrophils were incubated with 4C3 (2-100 μg/mL), r4C3 or separate IgG preparations from two healthy donors and four active GPA patients (200 μg/mL) for 45 minutes at 37° C. An irrelevant antibody (6H4, IgG1K, anti-ovalbumin) was used as a negative control. A combination of phorbol myristate acetate (PMA, 50 ng/mL) and calcium ionophore (ICa, 10 μM), both potent neutrophil activators, was used as a positive control for neutrophil activation. The reaction was stopped with iced EDTA PBS (1 mM) prior to measuring the fluorescence of DHR 123 by flow cytometry. For the neutralisation experiments, pre-treated primate neutrophils were first incubated with 4C3 (20 μg/mL) for 15 minutes at 37° C. and then separate IgG preparations of five patients active at the time of GPA diagnosis (IgG GPA, 200 μg/mL) were added for a further 45 minutes.

14.4 Degranulation and Adhesion of Neutrophils

Neutrophils were pre-activated with TNFα and stimulated with 4C3 (2 and 20 μg/mL), r4C3 (2 and 20 μg/mL), IgG from GPA patients (200 μg/mL) or PMA-ICa for 45 minutes at 37° C. After incubation, the cells were washed and labelled with CD63 FITC-coupled (degranulation) or labelled with CD11b VioBlue/CD18 FITC (BD Biosciences®, USA) antibodies (adhesion phenotype) for 20 minutes at 4° C. before being analysed by flow cytometry. The percentage of positive cells and the mean fluorescence intensity (MFI) were determined using FlowJo® software. Neutrophil degranulation was also evaluated by CatG release. The supernatants were collected and incubated with a cathepsin G (ABZ-TPFSGQ-YNO2 from GeneCust®) fluorescent substrate (Attucci S, et al. Measurement of free and membrane-bound cathepsin G in human neutrophils using new sensitive fluorogenic substrates. Biochem J. 2002 Sep. 15; 366(Pt 3):965-70. doi: 10.1042/BJ20020321. PMID: 12088507; PMCID: PMC1222843) for 30 minutes prior to reading the fluorescence spectrofluorimetry reading at 420 nm. The results are expressed as a report from ΔRFU compared to the normalized state from TNFα (basal degranulation).

Results

The first test consisted of evaluating the expression of membrane PR3 after incubation of neutrophils with 4C3 (FIG. 9). The 6H4 antibody, which is a non-relevant antibody, was used in comparison in the different tests and the PMA-Ionomycin calcium (PMA-ICa) solution was used as a positive control for neutrophil activation. Purified human neutrophils were pre-activated with TNFα for 15 minutes at 37° C. before being incubated for 1.5 hours with different concentrations of 6H4 and 4C3 antibodies (2, 20 and 100 μg/mL). At the end of incubation, membrane PR3 expression was analysed by flow cytometry with WGM2 antibody (murine anti-human PR3 antibody) (FIG. 9). The results showed that incubation of neutrophils with increasing doses of 4C3 leads to an increase in PR3 expression on the surface of neutrophils, whereas incubation with 6H4, regardless of the dose, has little or no effect (FIG. 9). The result was identical whether PR3 was revealed with a commercially available antibody or with 4C3-AF488.

The second functional test that was studied was the production of VSR, which was evaluated by DHR123-FITC marking by flow cytometry (FIGS. 10 and 11). For these experiments, total IgG from healthy donors or patients with GPA was also purified. Here, 4C3 was not able to induce ROS production, whereas separate IgG preparations from patients with active GPA (IgG GPA) led to a significant increase in ROS production. On the contrary, separate IgG preparations from healthy donors (IgG HD), obtained and used under the same conditions, did not induce a high production of ROS by pre-activated neutrophils (FIG. 10). It is interesting to note that the stimulation of neutrophils by purified IgG from patient P2 did not induce significant ROS production compared to purified IgG GPA from GPA patients at the time of diagnosis (FIG. 10). Furthermore, at a dose of 2 μg/mL, both 4C3 and 6H4 did not induce a significant increase in ROS production by neutrophils. By increasing the dose to 20 and 100 μg/mL, there was also no significant increase in the production of ROS by neutrophils, thus demonstrating that 4C3 in its whole form is not an activating antibody (FIG. 11).

The third test consisted of analysing neutrophil degranulation by measuring CD63 expression at the neutrophil membrane and cathepsin G activity in the neutrophil supernatant after incubation with antibodies (FIG. 12). The previously obtained result was confirmed since it was shown that there was a reciprocal absence of CD63 expression (FIG. 12A) and an absence of cathepsin G release in the presence of 4C3 or r4C3 at a dose of 2 and 20 μg/mL (FIG. 12B).

The fourth test consisted of analysing the expression of CD11b and CD18 (Mac1 complex) in order to explore the neutrophil adhesion phenotype after stimulation with 4C3 (FIG. 13). The presence of 4C3 did not induce an increase in CD11b/CD18 surface expression, whereas unmixed IgG preparations from healthy donors or patients with GPA at the time of diagnosis induced a significant upregulation of these two adhesion markers (FIGS. 13A and 13B). It should be noted that IgG from patient P2 induced an intermediate adhesion phenotype (FIGS. 13A and 13B).

All these results, obtained by functional tests on neutrophils, have shown that 4C3 and r4C3 antibodies do not activate human neutrophils.

Alongside these results, the effect of 4C3 derivatives was also studied. Before testing their functionality on neutrophils, the quality of these derived forms was verified by SDS-PAGE gel and Coomassie Blue staining (FIG. 14). For native 4C3, 6H4 and r4C3, a single migration band was observed around the molecular weight of 150 kDa, usually corresponding to the molecular weight of IgG1 (wells 2 to 4, FIG. 14). Quality and purity appear to be identical between 4C3, r4C3 and 6H4. Deposition of the Fab fragment revealed several bands: two bands around 50 kDa, one corresponding to the Fab fragment and the other to the Fc fragment which was not completely removed after purification on protein A-coupled resin and a third band around 25 kDa corresponding to the reduced Fab (6th well, FIG. 14). Deposition of the deglycosylated form revealed two bands: one slightly below 150 kDa corresponding to an IgG1 which had lost its glycosylation site and the other around 50 kDa corresponding to the Fc fragment (5th well, FIG. 14).

The expression of PR3 (FIG. 15A) and the neutrophil activation profile (FIGS. 15B and 16) in the presence of these derived forms were studied. No increase in membrane PR3 expression was observed after incubation of neutrophils with the Fab and F(ab′)₂ fragments of 4C3, unlike 4C3 in its whole form (FIG. 15A). In addition, the F(ab′)₂, Fab and degycosylated Fc (4C3 dFc) fragments did not induce SAR production by neutrophils at the 20 μg/mL dose (FIG. 15B).

Interestingly enough, it has been shown that r4C3 in its whole form is not capable of inducing a significant increase in SAR production and that this production is at the same level as that induced by TNFα: average MFI ration of the r4C3 condition=0.96±0.09 versus average ratio of the 4C3 condition=1.48±0.26 (p=0.0016) (FIG. 16), which corresponds to 0% increase in activation with r4C3.

In view of these results and with a view to obtaining a neutralising effect of the cANCA/PR3 interaction, the native and/or recombinant 4C3 in their whole form are the most interesting.

15. Neutralisation Tests with 4C3

In order to evaluate the blocking potential of this antibody (i.e. 4C3) on the activation of neutrophils induced by ANCA (anti-PR3 auto-antibodies), different strategies have been put in place:
functional tests were performed on neutrophils before and after incubation with the whole antibody or with its derivatives (Fab, F(ab′)₂, deglycosylated Fc, etc.); and
a dose effect was evaluated and different incubation kinetics were tested.

To develop these experiments to neutralise cANCA, it was decided to use sera from healthy donors (blood samples obtained from the EFS) and sera from GPA patients (sera obtained after anonymisation in the immunology laboratory of the CHRU de Tours) in order to purify the IgG and in particular the anti-PR3 IgG (cANCA) of GPA patients. Following the purification step on human serum G protein, the different fractions obtained (not retained, washes and elutions) were deposited on a gel. SDS-PAGE in order to check their quality and purity in comparison with the original serum (FIG. 17). This confirmed that the purification step of the sera on protein G allowed IgG to be obtained with the presence of a majority band at 150 kDa (FIG. 17) while the wash fractions contain other proteins such as albumin (band at 50 kDa).

After successfully purifying total IgG from sera, it was verified that among the purified IgG from GPA patients, anti-PR3 cANCA type IgG was present. By ELISA, it was found that the purified IgG batches from GPA patient sera contained at least 20 RU (Relative Unit)/mL of anti-PR3 IgG (light grey histograms, before and after purification, FIG. 18) in comparison with the calibrator (cal), 4C3 and the positive control (dark grey histograms, FIG. 18). In addition, no anti-PR3 IgG was detected with this technique in the purified IgG batches from healthy donor sera (black histograms, before and after purification, FIG. 18). Although this semi-quantitative assay confirmed the presence of anti-PR3 IgG in purified IgG from GPA patients (a representative experiment of 6 batches is shown), it did not reveal the true proportion.

Purified IgG was used at 200 μg/mL as described in the literature. The functionality of purified IgG was evaluated on their ability to induce ROS production in human neutrophils from healthy donors pre-activated by TNFα (as previously) and after 45 minutes incubation. Purified IgG from GPA patients significantly (p=0.0002; n=32) induced greater ROS production (mean MFI ratio=4.54±4.73) than purified IgG from healthy donors (mean MFI ratio=1.79±1.49) (FIG. 19). In addition, purified IgG from GPA patients and purified IgG from healthy donors induced significantly higher ROS production than for TNFα and condition 4C3 (FIG. 19). This result therefore demonstrated that purified IgG from GPA patients can be used as a neutrophil activator to test the neutralising potential of 4C3 and other derivatives.

In order to test the neutralising action of 4C3 on the production of ROS, it was chosen to first incubate neutrophils with 4C3 for 15 minutes and then add purified IgG from GPA patients for a further 45 minutes. The ROS production was then analysed as described above. It was found that 4C3 antibody at a concentration of 20 μg/mL does not activate neutrophils when used alone and quite unexpectedly, 4C3 antibody alone is also able to inhibit ROS activation induced by the presence of IgG from GPA patients (FIG. 20). Indeed, percentages of inhibition ranging from 36% to 71% were observed (average of 51% neutralisation over 4 separate experiments), which could be improved by increasing the amounts of 4C3 and/or modifying the incubation kinetics.

1.-13. (canceled)

14. A monoclonal antibody directed against neutrophil proteinase 3 represented by the sequence SEQ ID NO: 1,
said monoclonal antibody:
being specifically directed against a conformational epitope of said neutrophil proteinase 3; and
being capable of inhibiting by at least 30% the production of reactive oxygen derivatives by neutrophils,
said production of reactive oxygen derivatives being induced by the presence of autoantibodies directed against said neutrophil proteinase 3.

15. The monoclonal antibody according to claim 14, comprising:
a heavy chain comprising from its N-terminal to its end C-terminal:
a CDR1 having at least 80% identity with the sequence SEQ ID NO: 15;
a CDR2 having at least 80% identity with the sequence SEQ ID NO: 17; and
a CDR3 having at least 80% identity with the sequence SEQ ID NO: 19; and
a light chain comprising from its N-terminal to its end C-terminal:
a CDR1 having at least 80% identity with the sequence SEQ ID NO: 31;
a CDR2 having at least 80% identity with the sequence SEQ ID NO: 33; and
a CDR3 having at least 80% identity with the sequence SEQ ID NO: 35.

16. The monoclonal antibody according to claim 14, comprising:
a heavy chain comprising a variable region having at least 80% identity with the sequence SEQ ID NO: 7, with the proviso that said variable region of the heavy chain comprises from its N-terminal to its end C-terminal:
the CDR1 of sequence SEQ ID NO: 15;
the CDR2 of sequence SEQ ID NO: 17; and
the CDR3 of sequence SEQ ID NO: 19; and
a light chain comprising a variable region having at least 80% identity with the sequence SEQ ID NO: 25, with the proviso that said variable region of the light chain comprises from its N-terminal to its end C-terminal:
the CDR1 of sequence SEQ ID NO: 31;
the CDR2 of sequence SEQ ID NO: 33; and
the CDR3 of sequence SEQ ID NO: 35.

17. The monoclonal antibody according to claim 14, comprising:
a heavy chain comprising or consisting of a sequence having at least 80% identity with the sequence SEQ ID NO: 3, with the proviso that said heavy chain comprises the variable region of sequence SEQ ID NO: 7;
and
a light chain comprising or consisting of a sequence having at least 80% identity with the sequence SEQ ID NO: 21, with the proviso that said heavy chain comprises the variable region of sequence SEQ ID NO: 25.

18. A pharmaceutical composition comprising as active substance at least the monoclonal antibody according to claim 14 in combination with a pharmaceutically acceptable vehicle,
said monoclonal antibody being at a dose of 5 mg to 1,000 mg.

19. A method for the early treatment and/or prevention of relapse of granulomatosis with polyangiitis comprising the administration of the pharmaceutical composition according to claim 18.

20. The method according to claim 19, wherein said pharmaceutical composition is formulated to be administered by one of the following routes: oral, parenteral, injectable, topical, inhalation, subcutaneous, nasal or pulmonary.
